(19) United States
(12) Patent Application Publication
(10) Pub. No.: US 2010/0215604 A1
(43) Pub. Date: Aug. 26, 2010

(54) Cosmetic Compositions with Chitosan and Silicone Elastomers

(76) Inventors: Helga Van Flodrop, Hamburg (DE); Dirk Hentrich, Hamburg (DE)

Correspondence Address: Henkel Corporation, 10 Finderne Avenue, Bridgewater, NJ, 08807, US

(21) Appl. No.: 12/708,559

(22) Filed: Feb. 19, 2010

Related U.S. Application Data
(63) Continuation of application No. PCT/EP2008/060683, filed on Aug. 14, 2008.

(30) Foreign Application Priority Data
Aug. 21, 2007 (DE) 10 2007 039 519.3

(51) Int. Cl. A61K 8/89; A61Q 5/12; A61Q 5/02

(52) U.S. Cl. 424/70.2; 424/70.122; 510/122

(57) ABSTRACT

Hair treatment compositions comprising 0.05 to 5% by weight of at least one chitosan and/or chitosan derivative and 0.05 to 5% by weight of at least one elastomeric silane or siloxane with quaternary ammonium groups in the molecule advantageously incorporated into the hair treatment compositions, significantly improving fullness, volume and long-term hold.

### CROSS REFERENCE TO RELATED APPLICATIONS

The present application is a continuation of International Application No. PCT/EP2008/060683 filed 14 Aug. 2008, which claims the benefit of German Patent Application No. 10 2007 039 519.3, filed 21 Aug. 2007.

The present invention relates to hair treatment agents and their use. In particular, the present invention relates to hair cleaning compositions and hair conditioning compositions for improving the condition of hair.

Cosmetic treatment of skin and hair is an important component of human body care. Nowadays, human hair is treated in a variety of ways with hair cosmetic preparations. They include, for example, cleaning hair with shampoos, care and regeneration with rinses and cures, as well as bleaching, dyeing and styling hair with colorants, toners, permanent wave lotions and styling preparations. Among these, agents for changing or nuancing hair color play a prominent role.

The significance of care products with a long-lasting effect is growing, due not least to the serious stressing of hair by, for example, color-changing treatments or permanent waving, but also to shampooing and harmful environmental factors. These care products influence the natural structure and properties of the hair. Thus, after treatment with a care product, wet and dry combability of the hair and hold and volume of the hair can be optimized, or the hair can be protected against increased splitting.

Consequently, it has been common for some time to subject hair to a special after treatment. For this the hair is treated with special active substances, for example, quaternary ammonium salts or special polymers, usually in the form of a conditioner. Depending on the formulation, combability, hold and volume of the hair are improved and splitting rate reduced by this treatment.

So-called combination preparations have recently been developed to reduce the complexity of typical multistage methods, particularly where the preparations are directly applied by the user.

In addition to the usual components for cleaning the hair, for example, these preparations can also contain additional active substances previously reserved for hair after treatment preparations. Thus, the consumer saves an application step and at the same time, packaging costs are reduced because one less product is used.

Available active components, both for separate after treatment compositions and for combination preparations, generally act preferentially on the surface of the hair. Thus, active components are known which provide the hair with shine, hold, volume, better wet or dry combability or prevention of splitting. Just as important as the outward appearance of the hair, however, is the inner structural cohesion of the hair fibers, which can be seriously affected, in particular, by oxidative and reductive processes such as coloring and permanent waving.

However, known active components cannot adequately meet all requirements. Accordingly, there still remains a need for active substances or combinations of active substances for cosmetics with good caring properties and good biological degradability. There further remains the need for active care substances that can be incorporated without difficulty into known formulations, particularly in formulations containing dyes and/or electrolytes. In particular, active substances that lend the hair fullness and volume are characterized to a great extent in that the effects diminish over time and do not last over the total period between two hair treatments. This loss of fullness and volume—for example over the period of a working day—is not desirable by many consumers.

The present invention provides hair treatment agents that lend improved fullness and increased volume to hair treated with them. In particular, the hold of the hairstyle is improved for a longer period of time (long term hold), resulting in fullness and volume also being retained for a longer period of time.

It has now been found that certain combinations of active substances made of silicone and chitosan can be advantageously incorporated in hair treatment agents, and in addition to advantageous product and production characteristics, also significantly improve fullness, volume and long term hold.

The subject matter of the present invention in a first embodiment is hair treatment agents, comprising—based on their weight—
a) 0.05 to 5 wt. % of at least one chitosan and/or chitosan derivative, and
b) 0.05 to 5 wt. % of at least one elastomeric silane or siloxane containing quaternary ammonium groups in the molecule

In the context of the present invention, hair treatment agents include hair shampoos, hair conditioners, conditioning shampoos, hair sprays, hair rinses, hair cures, hair masks, hair tonics, permanent wave fixing solutions, hair dye shampoos, hair dyes, hair setting lotions, hair setting preparations, hair styling preparations, blow dry wave lotions, mousse, hair gels, hair waxes or their combinations. Preferred inventive compositions include shampoos, conditioners or hair tonics.

The inventive hair treatment agents comprise—based on their weight—0.05 to 5 wt. % of at least one chitosan and/or chitosan derivative.

Chitosans are biopolymers and are considered to be in the group of the hydrocolloids. From a chemical point of view, they are partially deacetylated chitins of different molecular weight.

Chitosan is manufactured from chitin, preferably from the shell remains of crustaceans, which are available in large quantities as a cheap raw material. The chitin is firstly deproteinated by adding bases, demineralized by adding mineral acids, and finally deacetylated by adding strong bases, wherein the molecular weights can vary over a wide spectrum. Those types are preferably employed that have an average molecular weight of 800,000 to 1,200,000 Dalton, a Brookfield viscosity (1 wt. % conc. in glycolic acid) 5,000 mPas or lower, a deacetylation degree in the range of 80 to 88% and an ash content of 0.3 wt. % or less.

In the scope of the invention, in addition to chitosans as typical cationic biopolymers, anionically, non-ionically or cationically derivatized chitosans can also be considered, such as carboxylated, succinylated, alkoxylated or quaternized products.

Inventively preferred hair treatment agents comprise neutralization products of chitosan with lactic acid and/or pyrrolidone carboxylic acid and/or nicotinic acid and/or hydroxy-iso-butyric acid and/or hydroxy-iso-valeric acid or mixtures of these neutralization products as the chitosan derivative(s).

According to the invention, further preferred hair conditioners comprise, based on their weight, 0.1 to 4.5 wt. %, preferably 0.25 to 4 wt. %, particularly preferably 0.5 to 3.5 wt. % more preferably 0.75 to 3 wt. % and particularly 1 to 2 wt. % of a neutralization product of chitosan with pyrrolidone carboxylic acid.

According to the invention, hair conditioners additionally comprise, based on their weight, 0.05 to 5 wt. % of at least one elastomeric silane or siloxane containing quaternary ammonium groups in the molecule. Elastomeric silanes or siloxanes are solid, but elastically deformable, and have a glass transition point that is below room temperature.

The inventively employable silicone elastomers are commercially available widely and are preferably incorporated in the form of emulsions into the inventive compositions. Particularly preferred inventive hair treatment agents comprise the elastomeric silane(s) or siloxane(s) having quaternary ammonium groups in the molecule in the form of an oil-in-water (O/W) or water-in-oil (W/O) emulsion or microemulsion, in which the silane or siloxane forms the oil phase.

It has been shown that a particularly good long-term hold with high fullness and high volume can be achieved with specific silicone elastomers. These specific silicone elastomers are quite particularly preferred in the context of the present invention as they produce, in combination with the chitosan (derivative), unusually good effects even at low added concentrations.

Particularly preferred hair treatment agents according to the invention accordingly include those wherein the elastomeric silane(s) or siloxane(s) having quaternary ammonium groups in the molecule are obtained from the reaction of
(i) an organic quaternary ammonium compound containing epoxide groups or halohydrin groups, with
(ii) a silane or siloxane containing amino groups, in the presence of
(iii) a branching agent, and
(iv) a surfactant, dispersed in
(v) an aqueous polar phase.

Preferred elastomeric silanes and/or siloxanes are obtained by reaction of the above mentioned components. Certain representatives of the individual substance groups are preferred and are described below:

Preferred organic quaternary ammonium compounds containing epoxide groups or halohydrin groups include glycidyl-trimethylammonium chloride and/or glycidyl-trimethylammonium bromide, as well as (3-chloro-2-hydroxypropyl)-trimethylammonium chloride, (3-chloro-2-hydroxypropyl)dimethyldodecyl-ammonium chloride, (3-chloro-2-hydroxypropyl)dimethyloctadecylammonium chloride, (3-chloro-2-hydroxy-propyl)trtmethylammonium bromide, (3-chloro-2-hydroxypropyl)dimethyldodecylammonium bromide, and (3-chloro-2-hydroxypropyl)dimethyloctadecylammonium bromide.

These compounds—the branching agents—are capable of reacting with free OH or NH₂ groups. In contrast to the branching agents, quaternary ammonium compounds containing epoxide groups or halohydrin groups are monofunctional, i.e., they do not branch the siloxanes.

Hair treatment agents are inventively preferred wherein the organic quaternary ammonium compound containing epoxide groups is chosen from glycidyl-trimethylammonium chloride and/or glycidyl-trimethylammonium bromide.

Hair treatment agents are likewise inventively preferred wherein the organic quaternary ammonium compound containing halohydrin groups is chosen from
(3-chloro-2-hydroxypropyl)trimethylammonium chloride,
(3-chloro-2-hydroxypropyl)dimethyldodecylammonium chloride
(3-chloro-2-hydroxypropyl)dimethyloctadecylammonium chloride,
(3-chloro-2-hydroxypropyl)trimethylammonium bromide,
(3-chloro-2-hydroxypropyl)dimethyldodecylammonium bromide, and
(3-chloro-2-hydroxypropyl)dimethyloctadecylammonium bromide.

Preferred siloxanes containing amino groups can be described by the general formula (SIL-I):

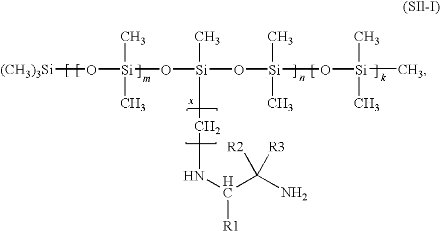

wherein indices k, m and n are chosen such that the siloxane contains 100 to 5000 silicon atoms. Indices k, m and n here preferably stand in a ratio to one another such that the —(CH₂)ₓ—NH—CH(R¹)—C(R²)(R³)—NH₂ groups make up 1 to 5 mol. %, preferably 1.5 to 4 mol. %, more preferably 2 to 3 mol. % and especially 2 to 2.5 mol. %. X represents a number 1, 2 or 3; and R¹, R² and R³ independently represent H or —CH₃.

Preferred —(CH₂)ₓ—NH—CH(R¹)—C(R²)(R³)—NH₂ groups include aminomethylaminoethyl groups, aminomethylaminopropyl groups, aminomethylaminoisopropyl groups, aminomethylaminobutyl groups, aminomethylaminoisobutyl groups, aminoethylaminoethyl groups, aminoethylaminopropyl groups, aminoethylaminoisopropyl groups, aminoethylaminobutyl groups, aminoethylaminoisobutyl groups, aminopropylaminoethyl groups, aminopropylaminopropyl groups, aminopropylaminoisopropyl groups, aminopropylaminobutyl groups, and aminopropylaminoisobutyl groups.

Particularly preferred siloxanes containing amino groups can be described by the formulas (SIL-Ia) to (SIL-If):

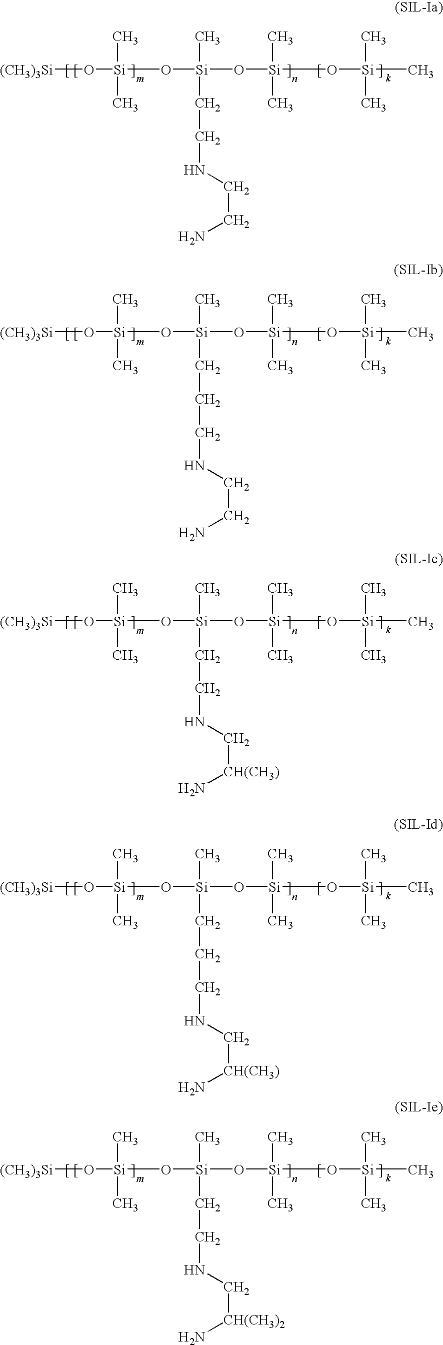

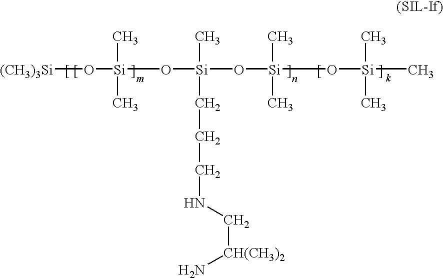

Siloxanes of the formula (SIL-Ie), which possess aminoethylaminoisobutyl groups, are particularly preferred.

Reaction of organic quaternary ammonium compounds containing epoxide groups or halohydrin groups with a silane or siloxane containing amino groups affords compounds in which a part of the amino groups has reacted with the epoxide group or halohydrin group. The reacting amino-functional group is converted into a quat group.

Preferred inventive hair treatment agents resulting from the preferred quaternary ammonium compounds mentioned further above containing epoxide groups or halohydrin groups are those wherein the elastomeric silane(s) or siloxane(s) containing quaternary ammonium groups in the molecule possess

—CH₂CH(OH)CH₂N⁺(CH₃)₃Cl⁻

as the quaternary ammonium group.

The reaction of the organic quaternary ammonium compound containing epoxide groups or halohydrin groups with a silane or siloxane containing amino groups preferably yields siloxanes that comprise moieties of the type

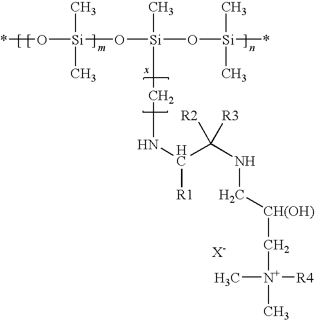

wherein m and n as well as x and R¹, R² and R³ are defined as above; X represent Cl or Br; and R⁴ is an alkyl group, preferably a methyl, dodecyl or octadecyl group.

Particularly preferably, X═Cl and R=methyl, so that preferred siloxanes comprise moieties of the type—

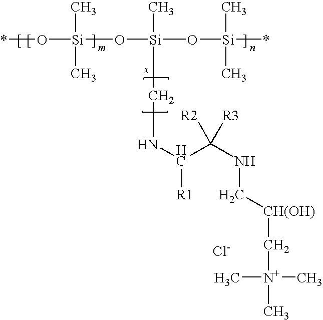

Taking into account that not all free amino groups react in the reaction, siloxanes of the formula (SIL-II) are obtained:

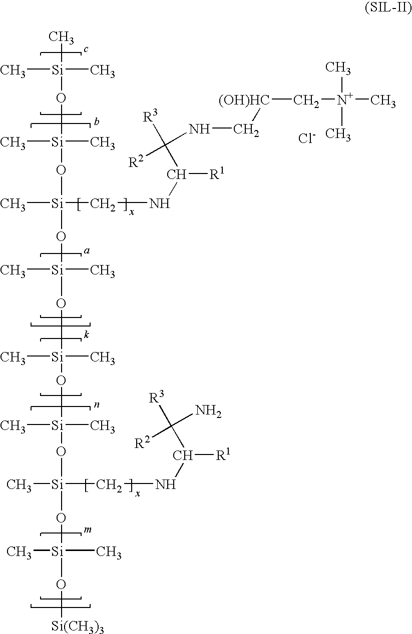

wherein m, n, k, a, b, c are chosen so that the siloxane contains 100 to 5000 silicon atoms. The indices preferably stand in a ratio to one another such that the reacted and unreacted —(CH₂)ₓ—NH—CH(R¹)—C(R²)(R³)—NH₂ groups make up 1 to 5 mol. %, preferably 1.5 to 4 mol. %, more preferably 2 to 3 mol. % and especially 2 to 2.5 mol. %. The degree of conversion, i.e., the number of —(CH₂)ₓ—NH—CH(R¹)—C(R²)(R³)—NH₂ groups that have reacted with the organic quaternary ammonium compound containing epoxide groups or halohydrin groups, is preferably 1 to 50% of all the —(CH₂)ₓ—NH—CH(R¹)—C(R²)(R³)—NH₂ groups, particularly preferably 5 to 40% and especially 10 to 30%.

Bearing in mind the preferred siloxanes (SIL-Ia) to (SIL-If), their reaction with the organic quaternary ammonium compound containing epoxide groups or halohydrin groups afford the preferred siloxanes (SIL-IIa) to (SIL-IIf):

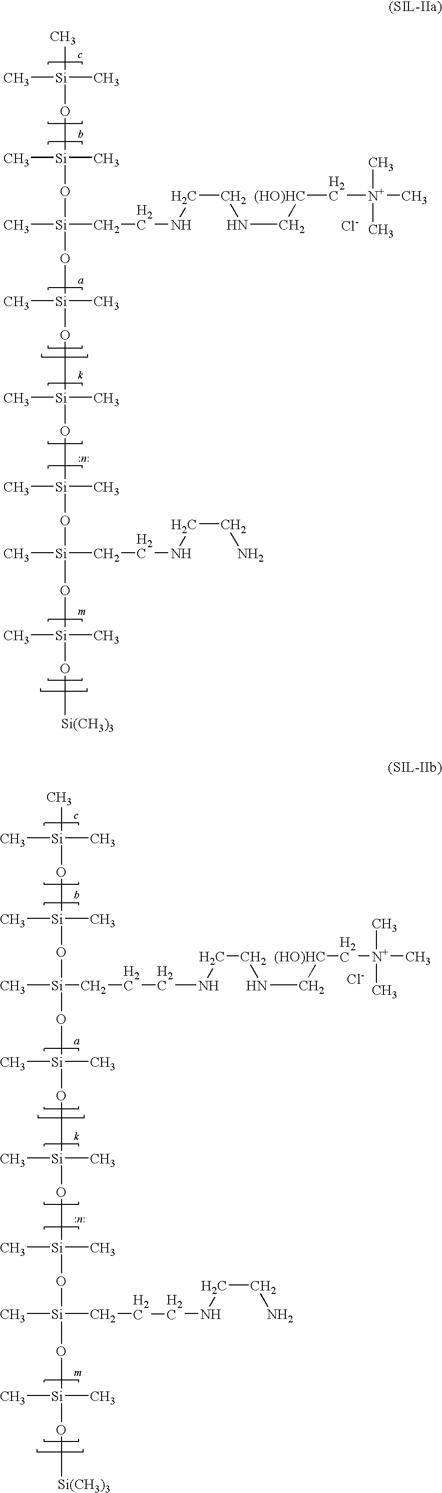

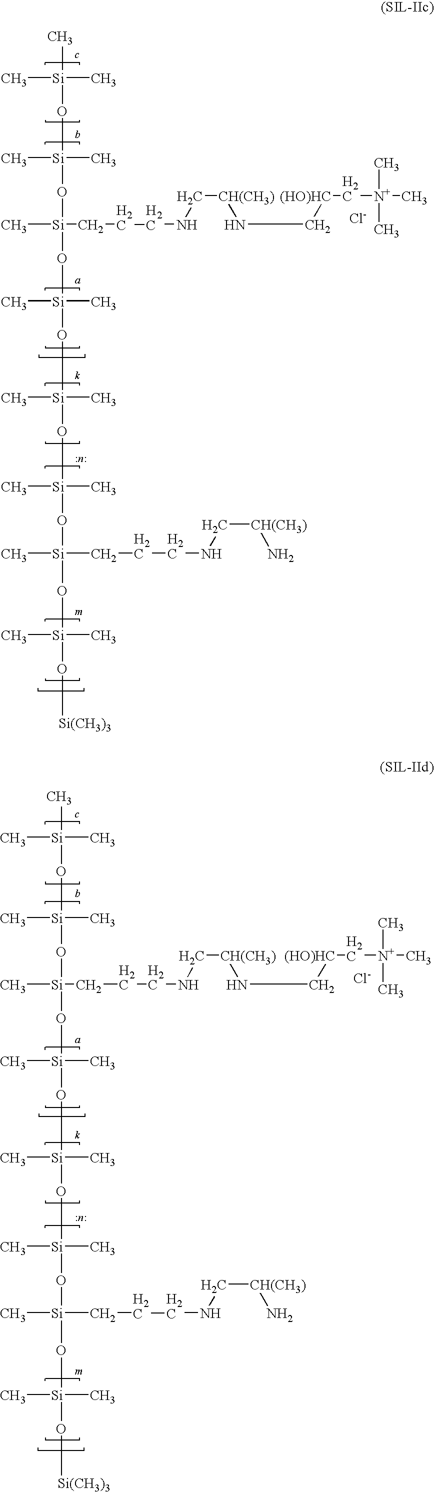

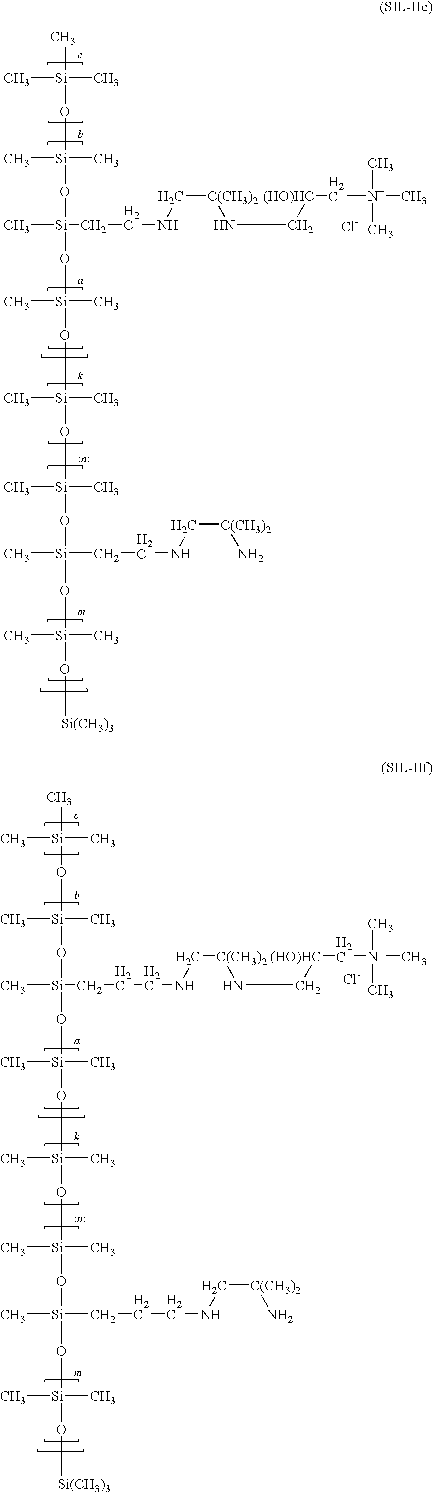

(SIL-IIf), siloxanes of the formula (SIL-IIe) being once again preferred.

A branching agent can also be included in the reaction mixture. This branching agent is preferably chosen from organic epoxides containing at least two epoxide groups, epoxy-functional silicones containing at least two epoxide groups, chlorohydrins, substituted di(meth)acrylates, unsubstituted di(meth)acrylates, oligo(meth)acrylates, substituted mono(meth)acrylates; hydroxyalkyl acrylates, and isocyanates.

Compounds containing two epoxide groups are particularly preferred; among these are butane diol diglycidyl ether

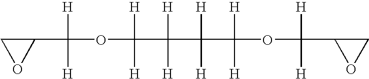

and polypropylene glycol diglycidyl ether

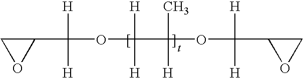

with t=1 to 100, preferably 1 to 50, particularly preferred.

In summary, hair treatment agents according to the invention include those wherein the branching agent is chosen from organic epoxides containing at least two epoxide groups, epoxy-functional silicones containing at least two epoxide groups, chlorohydrins, substituted di(meth)acrylates, unsubstituted di(meth)acrylates, oligo(meth)acrylates, substituted mono(meth)acrylates; hydroxyalkyl acrylates, and isocyanates.

Further, hair treatment agents according to the invention include those wherein the branching agent is chosen from butane diol diglycidyl ether and/or propylene glycol diglycidyl ether.

The reaction of the branching agent with the silane or siloxane containing amino groups affords compounds in which a part of the amino groups has reacted with the epoxide group or halohydrin group of the branching agent. Thus, the reacting amino-functional group of a siloxane becomes bonded to the reacting amino-functional group of another siloxane, thereby affording branched siloxanes, which comprise moieties of the type—

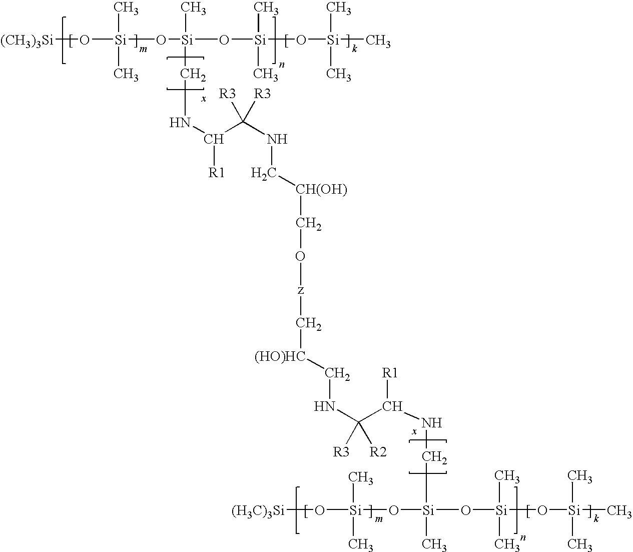

wherein m and n as well as x as well as R¹, R² and R³ are defined as above and Z stands for —(CH₂)₄O— or for —[CH₂—CH(CH₃)—O]ₜ, with t=1 to 100, preferably 1 to 50.

A preferred moiety Z is —(CH₂)₄—O—; as mentioned further above, x is preferably 2; R¹ is preferably —H; and R² and R³ are preferably methyl.

As a fraction of the amino groups of the siloxane remains converted, another fraction reacts with the branching agent, while another fraction reacts with the organic quaternary ammonium compound, resulting in siloxane cross-polymers containing amino-functional groups and quat groups. Addition of these silicone elastomers in the inventive hair treatment agents is preferred. Inventive silicone elastomers are particularly preferably added, which can be described by the following formula:

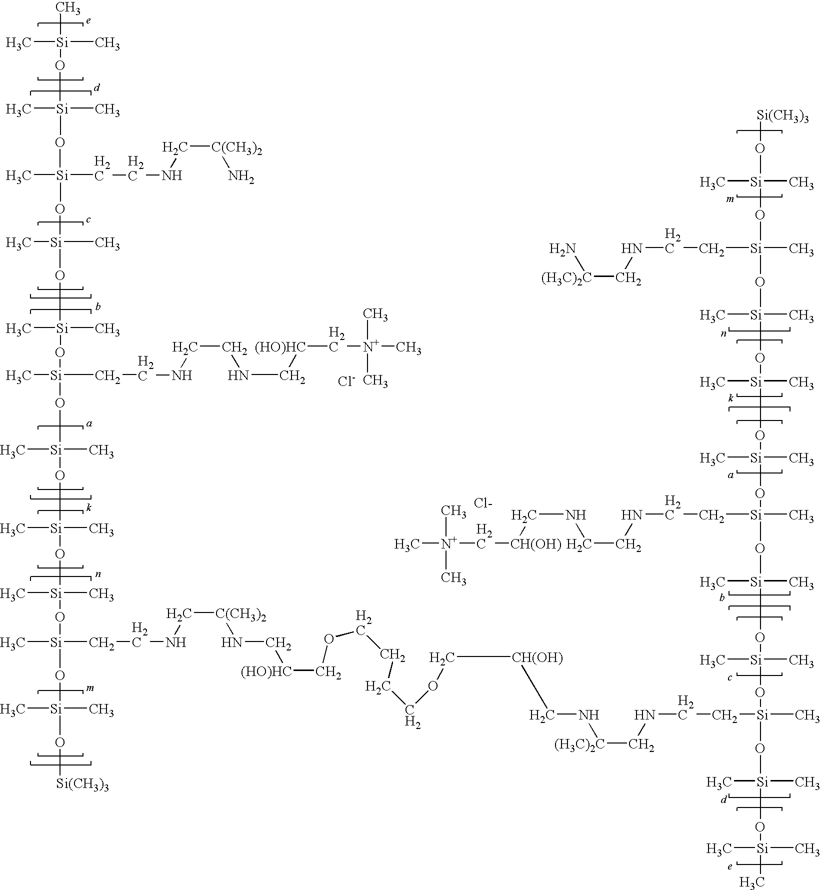

Moreover, the reaction mixture comprises at least one surfactant, such that the subsequent emulsion of the silicone elastomer likewise comprises surfactant. The surfactants described further below as possible ingredients of the inventive agent are also suitable ingredients of the reaction mixture.

The reaction mixture preferably comprises non-ionic surfactants, with non-ionic surfactants of the alkoxylated alcohol type preferred. Ethoxylated alcohols are particularly preferred and among these are once again those containing 18 to 18 EO groups, preferably containing 10 to 16 EO groups and especially containing 12 EO groups, wherein the cited degrees of ethoxylation represent average values. The alkyl chain of the ethoxylated alcohols has preferably 8 to 20 carbon atoms, wherein alkyl chains containing 10 to 18 carbon atoms are preferred and those containing 12 to 16 carbon atoms are particularly preferred. The tridecyl group is quite particularly preferred.

In summary, hair treatment agents according to the invention are preferred in which the surfactant in the emulsion of silicone elastomers is chosen from non-ionic surfactants, preferably from alkoxylated alcohols, particularly preferably from ethoxylated alcohols and especially from ethoxylated alcohols of the formula—

CₙH₂ₙ₊₁(OCH₂CH₂)_{X}OH

wherein n represents numbers between 8 and 20, preferably for 13; and x represents numbers from 8 to 18, preferably for 12

The aqueous polar phase of the silicone elastomer emulsion comprises water. In addition, non-aqueous polar solvents can be comprised, for example ethanol, n-propanol, isopropanol, diols such as ethylene glycol, propylene glycol, 1,6-hexane diol, 2-methyl-1,3-propane diol, triols such as glycerine esters such as triacetin and glycerine tripropionate etc. The aqueous silicone elastomer emulsion that is incorporated into the inventive hair treatment agents, comprises, based on its weight, 20 to 90 wt. %, preferably 30 to 80 wt. % and especially 50 to 70 wt. % water.

The inventive compositions can include additional active substances and auxiliaries. These are described below.

The inventive compositions preferably additionally comprise at least one emulsifier or surfactant, wherein surface active substances are designated as surfactants or as emulsifiers depending on their field of application, and are selected from anionic, cationic, zwitterionic, ampholytic and non-ionic surfactants and emulsifiers.

Inventively preferred hair treatment agents comprise, based on their weight, 0.5 to 70 wt. %, preferably 1 to 60 wt. % and particularly 5 to 25 wt. % of anionic and/or non-ionic and/or cationic and/or amphoteric surfactant(s).

Suitable anionic surfactants and emulsifiers for the inventive preparations include all anionic surface-active materials that are suitable for use on the human body. They are characterized by a water-solubilizing anionic group such as a carboxylate, sulfate, sulfonate or phosphate group and a lipophilic alkyl group containing about 8 to 30 carbon atoms. In addition, the molecule may contain glycol ether or polyglycol ether groups, ester, ether and amide groups as well as hydroxyl groups. Exemplary suitable anionic surfactants and emulsifiers can be in the form of sodium, potassium and ammonium as well as mono, di and trialkanolammonium salts with 2 to 4 carbon atoms in the alkanol group,
linear and branched fatty acids with 8 to 30 carbon atoms (soaps),
ether carboxylic acids of the formula R—O—(CH₂—CH₂O)ₓ—CH₂—COOH, in which R is a linear alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
linear alkane sulfonates with 8 to 24 carbon atoms,
linear alpha-olefin sulfonates with 8 to 24 carbon atoms
alpha-sulfo fatty acid methyl esters of fatty acids with 8 to 30 carbon atoms,
acylglutamates of the following Formula (I)

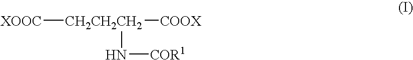

wherein R¹CO represents a linear or branched acyl group with 6 to 22 carbon atoms and 0, 1, 2 or 3 double bonds; and X represents hydrogen, an alkali and/or alkaline earth metal, ammonium, alkylammonium, alkanolammonium or glucammonium, for example acylglutamates that derive from fatty acids having 6 to 22, preferably 12 to 18 carbon atoms, such as C_{12/14}— or C_{12/18}— cocofatty acid, lauric acid, myristic acid, palmitic acid and/or stearic acid, particularly sodium N-cocoyl and sodium N-stearoyl-L-glutamate,
esters of a hydroxy-substituted di or tricarboxylic acid of the general formula (II)—

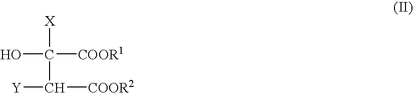

wherein X═H or is a —CH₂COOR group; Y═H or —OH, with the proviso that Y═H if X═—CH₂COOR; R, R¹ and R² are independently hydrogen, an alkali or alkaline earth metal cation, an ammonium group, the cation of an ammonium organic base or a group Z, which derives from a polyhydroxylated organic compound chosen from etherified (C₆-C₁₈)-alkylpolysaccharides having 1 to 6 monomeric saccharide units and/or etherified aliphatic (C₈-C₁₆)-hydroxyalkyl polyols having 2 to 16 hydroxyl groups, with the proviso that at least one of the groups R, R¹ and R² is a group Z,
esters of sulfosuccinic acid or of sulfosuccinates of the general formula (III),

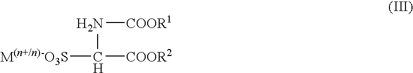

wherein M^{(n+/n)} for n=1 is a hydrogen atom, an alkali metal cation, an ammonium group or the cation of an ammonium organic base, and for n=2 an alkaline earth metal cation; and R¹ and R² independently represent hydrogen, an alkali or alkaline earth metal cation, an ammonium group, the cation of an ammonium organic base or a group Z, which derives from a polyhydroxylated organic compound selected from the group of the etherified (C₆-C₁₈)-alkylpolysaccharides having 1 to 6 monomeric saccharide units and/or the etherified aliphatic (C₆-C₁₈)-hydroxyalkyl polyols having 2 to 16 hydroxyl groups, with the proviso that at least one of the groups R¹ or R² is a group Z,
mono and dialkyl esters of sulfosuccinic acid containing 8 to 24 carbon atoms in the alkyl group and
mono alkyl polyoxyethyl esters of sulfosuccinic acid containing 8 to 24 carbon atoms in the alkyl group and 1 to 6 oxyethylene groups,
alkyl sulfates and alkyl polyglycol ether sulfates of formula R—(O—CH₂—CH₂)X—OSO₃H, in which R is preferably a linear alkyl group with 8 to 30 carbon atoms and x=0 or 1 to 12,
mixtures of surface-active hydroxy sulfonates according to DE-A-37 25 030,
esters of tartaric acid and citric acid with alcohols, which represent the addition products of about 2-15 molecules of ethylene oxide and/or propylene oxide on C₈₋₂₂ fatty alcohols,
alkyl- and/or alkenyl ether phosphates,
alkylene glycol esters of sulfated fatty acids, and
monoglyceride sulfates and monoglyceride ether sulfates.

Preferred anionic surfactants and emulsifiers include acyl glutamates, acyl isethionates, acyl sarcosinates and acyl taurates, each containing a linear or branched acyl group containing 6 to 22 carbon atoms and 0, 1, 2 or 3 double bonds, which in particularly preferred embodiments is chosen from an octanoyl, decanoyl, lauroyl, myristoyl, palmitoyl and stearoyl group, esters of tartaric acid, citric acid or succinic acid or their salts with alkylated glucose, in particular, products with the INCI-name disodium coco-glucoside citrate, sodium coco-glucoside tartrate and disodium coco-glucoside sulfosuccinate, alkylpolyglycol ether sulfates and ether carboxylic acids containing 8 to 18 carbon atoms in the alkyl group and up to 12 ethoxy groups in the molecule, mono and dialkyl esters of sulfosuccinic acid with 8 to 18 carbon atoms in the alkyl group and monoalkylpolyoxyethyl esters of sulfosuccinic acid containing 8 to 18 carbon atoms in the alkyl group and 1 to 6 ethoxy groups.

Zwitterionic surfactants and emulsifiers are designated as those surface-active compounds that carry at least one quaternary ammonium group and at least one —COO⁽⁻⁾ or —SO₃⁽⁻⁾ group in the molecule. Particularly suitable zwitterionic surfactants include betaines such as N-alkyl-N,N-dimethylammonium glycinates, for example, cocoalkyldimethylammonium glycinate, N-acylaminopropyl-N,N-dimethylammonium glycinates, for example, cocoacylaminopropyldimethylammonium glycinate, and 2-alkyl-3-carboxymethyl-3-hydroxyethyl imidazolines with 8 to 18 carbon atoms in each of the alkyl or acyl groups, as well as cocoacylaminoethylhydroxyethylcarboxymethyl glycinate. A preferred zwitterionic surfactant is the fatty acid amide derivative known under the INCI name Cocamidopropyl Betaine.

Ampholytic surfactants and emulsifiers include such surface-active compounds that apart from a C₈-C₂₂ alkyl or acyl group, comprise at least one free amino group and at least one COOH or SO₃H group, and are able to form internal salts. Examples of suitable ampholytic surfactants include N-alkylglycines, N-alkylamino propionic acids, N-alkylamino butyric acids, N-alkylimino dipropionic acids, N-hydroxyethyl-N-alkylamidopropylglycine, N-alkyltaurines, N-alkylsarcosines, 2-alkylamino propionic acids and alkylamino acetic acids, each with about 8 to 24 carbon atoms in the alkyl group. Particularly preferred ampholytic surfactants include N-cocoalkylamino propionate, cocoacylaminoethylamino propionate and C₁₂-C₁₈ acyl sarcosine.

Non-ionic surfactants and emulsifiers comprise, for example, a polyol group, a polyalkylene glycol ether group or a combination of polyol and polyglycol ether groups as the hydrophilic group. Exemplary compounds of this type include—
addition products of 2 to 50 moles ethylene oxide and/or 0 to 5 moles propylene oxide to linear and branched fatty alcohols containing 8 to 30 carbon atoms, to fatty acids with 8 to 30 carbon atoms, and to alkyl phenols with 8 to 15 carbon atoms in the alkyl group,
C₁₂-C₃₀ fatty acid mono- and diesters of addition products of 1 to 30 moles ethylene oxide on polyols containing 3 to 6 carbon atoms, especially glycerine,
addition products of 5 to 60 moles ethylene oxide on castor oil and hydrogenated castor oil,
polyol fatty acid (partial) esters such as Hydagen® HSP (Cognis) or Sovermol® types (Cognis), in particular, saturated C₈₋₃₀ fatty acids,
alkoxylated triglycerides,
alkoxylated fatty acid alkyl esters,
amine oxides,
fatty acid alkanolamides, fatty acid-N-alkylglucamides, and fatty amines, as well as their ethylene oxide or polyglycerine addition products,
sorbitol esters of fatty acids and addition products of ethylene oxide to sorbitol esters of fatty acids such as polysorbates,
sugar esters of fatty acids and methylglucoside fatty acid esters as well as their ethylene oxide or polyglycerine addition products,
alkyl polyglycosides corresponding to the general formula RO—(Z)ₓ wherein R stands for alkyl, Z for sugar and x for the number of sugar units. Such alkyl polyglycosides are particularly preferred wherein R consists substantially of—
   C₈ and C₁₀ alkyl groups,
   C₁₂ and C₁₄ alkyl groups,
   C₈ to C₁₆ alkyl groups, or
   C₁₂ to C₁₆ alkyl groups, or
   C₁₄ to C₁₈ alkyl groups.

Any mono- or oligosaccharide can be employed as the sugar building block Z. Usually, sugars with 5 or 6 carbon atoms as well as the corresponding oligosaccharides are used. Such sugars include glucose, fructose, galactose, arabinose, ribose, xylose, lyxose, allose, altrose, mannose, gulose, idose, talose and sucrose. Preferred sugar building blocks include glucose, fructose, galactose, arabinose and sucrose; and glucose is particularly preferred.

The inventively useable alkyl polyglycosides comprise on average 1.1 to 5 sugar units. Alkyl polyglycosides with x-values of 1.1 to 2.0 are preferred. Alkyl polyglycosides with x-values of 1.1 to 1.8 are quite particularly preferred. Examples include—
mixtures of alkyl (oligo)glucosides and fatty alcohols (e.g., Montanov® 68),
sterols, for example, ergosterol, stigmasterol, sitosterol and mycosterols,
phospholipids, for example, lecithin or phosphatidyl cholines,
polyglycerines and polyglycerine derivatives such as polyglycerine poly-12-hydroxystearate (Dehymuls® PGPH) or triglycerine diisostrearate (Lameform® TGI), and
alkoxylated polydialkylsiloxanes (INCI name: Dimethicone Copolyol).

Alkyl polyglycosides, optionally in a mixture with fatty alcohols, alkoxylated polydialkylsiloxanes, alkylene oxide addition products to saturated, linear fatty alcohols and fatty acids, each with 2 to 30 moles ethylene oxide per mole fatty alcohol or fatty acid, have proved to be preferred non-ionic surface active substances.

According to the invention, cationic surfactants of the type quaternary ammonium compounds, the esterquats and the amido amines can likewise be used. Preferred quaternary ammonium compounds are ammonium halides, especially chlorides and bromides, such as alkyltrimethylammonium chlorides, dialkyldimethylammonium chlorides and trialkylmethylammonium chlorides. The long alkyl chains of these surfactants preferably have 10 to 18 carbon atoms, such as e.g. in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are those imidazolium compounds known under the INCI names Quaternium-27 and Quaternium-83.

Particularly preferred inventive compositions comprise fatty alcohol(s) and/or fatty alcohol alkoxylate(s), preferably C₁₂₋₂₂ fatty alcohol(s) and/or C₁₂₋₂₂ fatty alcohol ethoxylate(s) with 10 to 30 EO units, particularly preferably C₁₆₋₁₈ fatty alcohol(s) and/or C₁₆₋₁₈ fatty alcohol ethoxylate(s) with 12 to 20 EO units, preferably in amounts of 5 to 20 wt. %, preferably from 7.5 to 17.5 wt. % and especially from 10 to 15 wt. %, each based on the weight of the composition.

In summary, inventive hair treatment agents are preferred that comprise, based on their weight, 0.1 to 20 wt. %, preferably 0.25 to 17.5 wt. % and particularly 5 to 15 wt. % of anionic surfactant(s), particularly preferably fatty alcohol ether sulfates of the formula—

H₃C—(CH₂MOCH₂CH₂)_{K}—OSO₃⁻M⁺

wherein n represents values of 5 to 21, preferably from 7 to 19, particularly preferably from 9 to 17 and especially from 11 to 13; k represents values of 1, 2, 3, 4, 5, 6, 7, 8, 9 or 10, preferably 1, 2 or 3 and especially 2; and M represents a cation from the group Na⁺, K⁺NH₄⁺, ½Mg²⁺, ½Zn²⁺, preferably Na⁺.

Preferred inventive hair treatment agents further comprise amphoteric surfactant(s), preferably from the group of—
N-alkylglycines,
N-alkylpropionic acids,
N-alkylamino butyric acids,
N-alkylimino dipropionic acids,
N-hydroxyethyl-N-alkylamido propylglycines,
N-alkyltaurines,
N-alkylsarcosines,
2-alkylamino propionic acids each with about 8 to 24 carbon atoms in the alkyl group,
2-alkylamino acetic acids each with about 8 to 24 carbon atoms in the alkyl group,
N-cocoalkylamino propionate,
cocoacylaminoethylamino propionate,
C₁₂-C₁₈ acylsarcosine,
N-alkyl-N,N-dimethylammonium glycinates, for example, cocoalkyldimethylammonium glycinate,
N-acyl-aminopropyl-N,N-dimethylammonium glycinates, for example, cocoacylaminopropyldimethylammonium glycinate,
2-alkyl-3-carboxymethyl-3-hydroxyethyl-imidazolines each with 8 to 18 carbon atoms in the alkyl or acyl group,
cocoacylaminoethylhydroxyethylcarboxymethyl glycinate,
compounds known under the INCI name cocamidopropyl betaine, and
compounds known under the INCI name disodium cocamphodiacetate,
   wherein preferred compositions comprise the amphoteric surfactant(s) in quantities from 1 to 15 wt. %, preferably from 2.5 to 12 wt. % and especially from 5 to 10 wt. %, each based on total weight of the composition.

The inventive compositions can also include as an optional ingredient 0.01 to 10 wt. % of at least one polymer from the group of cationic and/or amphoteric polymers.

Cationic or amphoteric polymers are understood to mean polymers that have groups in their main chain or side chain that can be “temporarily” or “permanently” cationic. “Permanently cationic” according to the invention, refers to those polymers which, independently of the pH of the medium, have a cationic group. These are generally polymers comprising a quaternary nitrogen atom, for example, in the form of an ammonium group. Preferred cationic groups include quaternary ammonium groups. In particular, those polymers in which the quaternary ammonium groups are bonded through a C₁₋₄ hydrocarbon group to a polymer backbone of acrylic acid, methacrylic acid or their derivatives, have proved to be particularly suitable.

In addition to or in place of cationic polymers, the inventive compositions can also comprise amphoteric polymers. They can also have at least one negatively charged group in the molecule and are also called zwitterionic polymers.

The polymer or polymers is/are preferably employed in narrow quantitative ranges. According to the invention, preferred compositions comprise, based on their weight, 0.05 to 7.5 wt. %, preferably 0.1 to 5 wt. %, particularly preferably 0.2 to 3.5 wt. % and particularly 0.25 to 2.5 wt. % amphoteric polymer(s).

Independently of whether amphoteric polymers are found in the compositions or not, further preferred inventive compositions comprise, based on their weight, 0.05 to 7.5 wt. %, preferably 0.1 to 5 wt. %, particularly preferably 0.2 to 3.5 wt. % and particularly 0.25 to 2.5 wt. % cationic polymer(s).

Inventively preferred employable cationic polymers are described below: Homopolymers of the general formula (G1-I)—

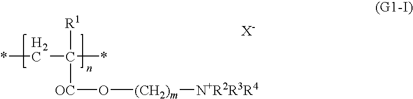

wherein R¹=—H or —CH₃; R², R³ and R⁴ are independently C₁₋₄ alkyl, -alkenyl or -hydroxyalkyl groups; m=1, 2, 3 or 4; n is a natural number; and X⁻ is a physiologically compatible organic or inorganic anion, as well as copolymers, essentially consisting of the monomer units listed in formula (G1-I) as well as non-ionic monomer units, are particularly preferred cationic polymers. Regarding these polymers, those that are preferred in accordance with the invention meet at least one of the following conditions:
R¹ represents a methyl group,
R², R³ and R⁴ represents methyl groups, and
m is the value 2.

Exemplary physiologically compatible counter ions X⁻ include halide ions, sulfate ions, phosphate ions, methosulfate ions as well as organic ions such as lactate, citrate, tartrate and acetate ions. Halide ions are preferred, particularly chloride.

A particularly suitable homopolymer is the optionally crosslinked poly(methacryloyloxyethyltrimethylammonium chloride) with the INCI name Polyquaternium-37. Such products are commercially available under the trade names Rheocare® CTH (Cosmetic Rheologies) and Synthalen® CR (Ethnichem). Crosslinking can be effected, when desired, with the help of olefinically polyunsaturated compounds, for example divinylbenzene, tetraallyloxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably employed in the form of a non-aqueous polymer dispersion that should have a polymer content of not less than 30 wt. %. Such polymer dispersions are commercially available under the names Salcare® SC 95 (ca. 50% polymer content, additional components: mineral oil (INCI name: Mineral Oil) and tridecyl-polyoxypropylene polyoxyethylene ether (INCI name: PPG-1-Trideceth-6)) and Salcare® SC 96 (ca. 50% polymer content, additional components: mixture of diesters of propylene glycol with a mixture of caprylic acid and capric acid (INCI name: Propylene Glycol Dicaprylate/Dicaprate) and tridecyl-polyoxypropylene polyoxyethylene ether (INCI name: PPG-1-Trideceth-6)).

Copolymers with monomer units according to formula (G1-1) preferably comprise acrylamide, methacrylamide, C₁₋₄ alkyl esters of acrylic acid and C₁₋₄ alkyl esters of methacrylic acid as the non-ionic monomer units. Acrylamide is particularly preferred among these non-ionic monomers. These copolymers can also be crosslinked, as in the case of the above described homopolymers. An inventively preferred copolymer is the crosslinked acrylamide/methacryloyloxyethyltrimethylammonium chloride copolymer. Such copolymers, in which the monomers are present in a weight ratio of about 20:80, are commercially available as a ca. 50% conc. non-aqueous polymer dispersion named Salcare® SC 92.

Further preferred cationic polymers include, for example—
quaternized cellulose derivatives, commercially available under the names Celquat® and Polymer JR®. The compounds Celquat® H 100, Celquat® L 200 and Polymer JR®400 are preferred quaternized cellulose derivatives,
cationic alkyl polyglycosides according to DE-PS 44 13 686,
cationic honey (e.g., the commercial product Honeyquat® 50),
cationic guar derivatives, such as in particular the products marketed under the trade names Cosmedia® Guar and Jaguar®,
polymeric dimethyl diallyl ammonium salts and their copolymers with esters and amides of acrylic acid and methacrylic acid. The commercially available products Merquat® (Merquat®100 (poly(dimethyl diallyl ammonium chloride)) and Merquat®550 (dimethyl diallyl ammonium chloride-acrylamide copolymer) are examples of such cationic polymers,
copolymers of vinyl pyrrolidone with quaternized derivatives of dialkylaminoalkyl acrylate and dialkylaminoalkyl methacrylate, such as vinyl pyrrolidone-dimethylaminoethyl methacrylate copolymers quaternized with diethyl sulfate. Such compounds are commercially available under the trade names Gafquat® 734 and Gafquat® 755,
vinyl pyrrolidone-vinyl imidazolium methochloride copolymers, available under the trade names Luviquat® FC 370, FC 550, FC 905 and HM 552,
quaternized polyvinyl alcohol, as well as the known polymers containing quaternary nitrogen atoms in the main polymer chain, and
Polyquaternium 2, Polyquaternium 17, Polyquaternium 18 and Polyquaternium 27.

Polymers designated as Polyquaternium-24 (commercially available as, for example, Quatrisoft® LM 200) can also be employed as cationic polymers. Copolymers of vinyl pyrrolidone are also usable according to the invention, such as the commercially available products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Cationic protein hydrolyzates can also be employed as the cationic polymers, wherein preferred compositions comprise one or more cationic protein hydrolyzates from the group Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Pro-tein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

In summary, inventively preferred hair treatment agents comprise, based on their weight, 0.05 to 7.5 wt. %, preferably 0.1 to 5 wt. %, particularly preferably 0.2 to 3.5 wt. % and particularly 0.25 to 2.5 wt. % cationic polymer(s), wherein preferred cationic polymer(s) are chosen from
poly(methacryloyloxyethyltrimethylammonium chloride) (INCI: Polyquaternium-37); and/or
quaternized cellulose derivatives (INCI: Polyquaternium 10); and/or
cationic alkyl polyglycosides; and/or
cationized honey; and/or
cationic guar derivatives; and/or
polymeric dimethyldiallylammonium salts and their copolymers with esters and amides of acrylic acid and methacrylic acid; and/or
copolymers of vinyl pyrrolidone with quaternized derivatives of dialkylaminoalkyl acrylate and dialkylaminoalkyl methacrylate; and/or
vinyl pyrrolidone-vinylimidazolium methochloride copolymers; and/or
quaternized polyvinyl alcohol; and/or
Polyquaternium 2; and/or
Polyquaternium-7; and/or
Polyquaternium 17; and/or
Polyquaternium 18; and/or
Polyquaternium 24; and/or
Polyquaternium 27.

In addition to or in place of the cationic polymers, the inventive compositions can also comprise amphoteric polymers. In the context of the present invention, preferred employable amphoteric polymers are essentially composed of—

A) Monomers with quaternary ammonium groups of the general Formula (Z-I)—

R¹—CH═CR²—CO—Z—(CₙH₂ₙ)—N(⁺)R³R⁴R⁵A⁻ (Z-1)
wherein R¹ and R² independently are hydrogen or a methyl group; and R³, R⁴ and R⁵ independently represent alkyl groups with 1 to 4 carbon atoms; Z represents an NH-group or an oxygen atom; n represents a whole number from 2 to 5; and A⁽⁻⁾ represents the anion of an organic or inorganic acid, and
   B) monomers of carboxylic acids of the general Formula (Z-II)—

R⁶—CH═CR⁷—COOH (Z-II)
wherein R⁶ and R⁷ independently are hydrogen or methyl groups.

Suitable starting monomers include dimethylaminoethyl acrylamide, dimethylaminoethyl methacrylamide, dimethylaminopropyl acrylamide, dimethylaminopropyl methacrylamide and diethylaminoethyl acrylamide, if Z is an NH group, or dimethylaminoethyl acrylate, dimethylaminoethyl methacrylate and diethylaminoethyl acrylate, if Z is an oxygen atom.

The monomers comprising a tertiary amino group are then quaternized in the usual manner, wherein methyl chloride, dimethyl sulfate or diethyl sulfate are particularly suitable as the alkylation reagents. The quaternization reaction can be made in aqueous solution or in a solvent.

Advantageously, those monomers of formula (Z-I) are used, which are derivatives of acrylamide or methacrylamide. In addition, those monomers that comprise halide, methoxysulfate or ethoxysulfate ions as the counter ions are preferred. Those monomers of formula (Z-I), in which R³, R⁴ and R⁵ are methyl groups, are likewise preferred.

Acrylamidopropyltrimethylammonium chloride is a quite particularly preferred monomer of formula (Z-I).

Acrylic acid, methacrylic acid, crotonic acid and 2-methylcrotonic acid are suitable as the monomeric carboxylic acid of formula (Z-II). Acrylic acid or methacrylic acid, in particular acrylic acid, are preferably employed.

The inventively employable zwitterionic polymers are manufactured from monomers of formulas (Z-I) and (Z-II) according to known polymerization processes. Polymerization can be made in either aqueous or aqueous alcoholic solution. Alcohols containing 1 to 4 carbon atoms, preferably isopropanol, can be used as the alcohol, which simultaneously act as polymerization regulators. However, other components can also be added to the monomer solution, for example, formic acid or mercaptans such as thioethanol and thioglycolic acid. Polymerization is initiated by radical-forming substances. For this, redox systems and/or thermally decomposing radical sources of the azo compound type such as azoisobutyric acid nitrile, azo-bis(cyclopentanoic acid) or azo-bis(amidinopropane) dihydrochloride can be used. Combinations of hydrogen peroxide, potassium or ammonium peroxodisulfate, as well as tertiary butyl hydroperoxide are suitable redox systems, with sodium sulfite, sodium dithionite or hydroxylamine hydrochloride as the reductive components.

Polymerization can be carried out isothermally or under adiabatic conditions, wherein, depending on concentration ratios, the temperature range for the reaction process can vary from 20 to 200° C. due to the heat of reaction of polymerization, and the reaction possibly has to be carried out under the resulting overpressure. Preferably, the reaction temperature is from 20 to 100° C.

During copolymerization, the pH can vary over a wide range. Advantageously, polymerization is carried out at low pH; however, pH values above neutral are also possible. After polymerization, the pH is adjusted to from 5 to 10, preferably 6 to 8, with an aqueous base, (e.g., sodium hydroxide, potassium hydroxide or ammonium hydroxide). Further details of the polymerization process can be found in the Examples.

Those polymers possessing an excess of monomers of formula (Z-I) over the monomers of formula (Z-II) have proven to be particularly effective. Accordingly, it is inventively preferred to use polymers consisting of monomers according to formula (Z-I) and formula (Z-II) in a molar ratio of 60:40 to 95:5, particularly 75:25 to 95:5.

Compositions are inventively preferred wherein the amphoteric polymer(s) include monomers A) and B), wherein A) and B) are chosen from—

A) Monomers having quaternary ammonium groups of the general Formula (Z-I)—

R¹—CH═CR²—CO—Z—(CₙH₂ₙ)—N(⁺R³R⁴R⁵A⁻ (Z1)
in which R¹ and R² independently are hydrogen or a methyl group; R³, R⁴ and R⁵ independently are alkyl groups with 1 to 4 carbon atoms; Z represents an NH-group or an oxygen atom; n represents a whole number from 2 to 5; and A⁽⁻⁾ is the anion of an organic or inorganic acid; and
   B) monomers of carboxylic acids of the general Formula (Z-II)—

R⁶—CH═CR⁷—COOH (Z-II)
wherein R⁶ and R⁷ independently are hydrogen or methyl groups.

Amphoteric polymers utilized in the inventive agents particularly preferably comprise monomers from the group of the acrylamides and/or methacrylamides containing alkylammonium groups. Acrylic acid and/or methacrylic acid and/or crotonic acid and/or 2-methylcrotonic acid have proven to be successful as anionic group-containing monomers that are additionally comprised in the polymers.

In summary, agents according to the invention are preferred, in which the amphoteric polymer(s) are copolymers of at least one of the monomers—
trimethylammonium ethylacrylamide; and/or,
trimethylammonium ethylmethacrylamide; and/or
trimethylammonium propylacrylamide; and/or
trimethylammonium propylmethacrylamide; and/or
trimethylammonium ethylacrylamide; and/or
trimethylammonium ethyl acrylate; and/or
trimethylammonium ethyl methacrylate; and/or
trimethylammonium ethyl acrylate; and/or
ethyldimethylammonium ethylacrylamide; and/or,
ethyldimethylammonium ethylmethacrylamide; and/or
ethyldimethylammonium propylacrylamide; and/or
ethyldimethylammonium propylmethacrylamide; and/or
ethyldimethylammonium ethylacrylamide; and/or
ethyldimethylammonium ethyl acrylate; and/or
ethyldimethylammonium ethyl methacrylate; and/or
ethyldimethylammonium ethyl acrylate;
   with at least one of the monomers being
acrylic acid; and/or
methacrylic acid; and/or
crotonic acid; and/or
2-methylcrotonic acid.

Particularly preferred amphoteric polymers according to the invention include copolymers of—
trimethylammonium ethylacrylamide with acrylic acid,
trimethylammonium ethylacrylamide with methacrylic acid,
trimethylammonium ethylacrylamide with crotonic acid,
trimethylammonium ethylacrylamide with 2-methyl-crotonic acid,
trimethylammonium ethylmethacrylamide with acrylic acid,
trimethylammonium ethylmethacrylamide with methacrylic acid,
trimethylammonium ethylmethacrylamide with crotonic acid,
trimethylammonium ethylmethacrylamide with 2-methyl-crotonic acid,
trimethylammonium propylacrylamide with acrylic acid,
trimethylammonium propylacrylamide with methacrylic acid,
trimethylammonium propylacrylamide with crotonic acid,
trimethylammonium propylacrylamide with 2-methyl-crotonic acid,
trimethylammonium propylmethacrylamide with acrylic acid,
trimethylammonium propylmethacrylamide with methacrylic acid,
trimethylammonium propylmethacrylamide with crotonic acid,
trimethylammonium propylmethacrylamide with 2-methyl-crotonic acid,
trimethylammonium ethylacrylamide with acrylic acid,
trimethylammonium ethylacrylamide with methacrylic acid,
trimethylammonium ethylacrylamide with crotonic acid,
trimethylammonium ethylacrylamide with 2-methyl-crotonic acid,
trimethylammonium ethyl acrylate with acrylic acid,
trimethylammonium ethyl acrylate with methacrylic acid,
trimethylammonium ethyl acrylate with crotonic acid,
trimethylammonium ethyl acrylate with 2-methyl-crotonic acid,
trimethylammonium ethyl methacrylate with acrylic acid,
trimethylammonium ethyl methacrylate with methacrylic acid,
trimethylammonium ethyl methacrylate with crotonic acid,
trimethylammonium ethyl methacrylate with 2-methyl-crotonic acid,
trimethylammonium ethyl acrylate with acrylic acid,
trimethylammonium ethyl acrylate with methacrylic acid,
trimethylammonium ethyl acrylate with crotonic acid,
trimethylammonium ethyl acrylate with 2-methyl-crotonic acid,
ethyldimethylammonium ethylacrylamide with acrylic acid,
ethyldimethylammonium ethylacrylamide with methacrylic acid,
ethyldimethylammonium ethylacrylamide with crotonic acid,
ethyldimethylammonium ethylacrylamide with 2-methyl-crotonic acid,
ethyldimethylammonium ethylmethacrylamide with acrylic acid,
ethyldimethylammonium ethylmethacrylamide with methacrylic acid,
ethyldimethylammonium ethylmethacrylamide with crotonic acid,
ethyldimethylammonium ethylmethacrylamide with 2-methyl-crotonic acid,
ethyldimethylammonium propylacrylamide with acrylic acid,
ethyldimethylammonium propylacrylamide with methacrylic acid,
ethyldimethylammonium propylacrylamide with crotonic acid,
ethyldimethylammonium propylacrylamide with 2-methyl-crotonic acid,
ethyldimethylammonium propylmethacrylamide with acrylic acid,
ethyldimethylammonium propylmethacrylamide with methacrylic acid,
ethyldimethylammonium propylmethacrylamide with crotonic acid,
ethyldimethylammonium propylmethacrylamide with 2-methyl-crotonic acid,
ethyldimethylammonium ethylacrylamide with acrylic acid,
ethyldimethylammonium ethylacrylamide with methacrylic acid,
copolymers of ethyldimethylammonium ethylacrylamide with crotonic acid,
ethyldimethylammonium ethylacrylamide with 2-methyl-crotonic acid,
ethyldimethylammonium ethyl acrylate with acrylic acid,
ethyldimethylammonium ethyl acrylate with methacrylic acid,
ethyldimethylammonium ethyl acrylate with crotonic acid,
ethyldimethylammonium ethyl acrylate with 2-methyl-crotonic acid,
ethyldimethylammonium ethyl methacrylate with acrylic acid,
ethyldimethylammonium ethyl methacrylate with methacrylic acid,
ethyldimethylammonium ethyl methacrylate with crotonic acid,
ethyldimethylammonium ethyl methacrylate with 2-methyl-crotonic acid,
ethyldimethylammonium ethyl acrylate with acrylic acid,
ethyldimethylammonium ethyl acrylate with methacrylic acid,
ethyldimethylammonium ethyl acrylate with crotonic acid, and
ethyldimethylammonium ethyl acrylate with 2-methyl-crotonic acid.

Agents according to the invention advantageously can comprise one or more amino acids as additional ingredients. Inventively particularly preferred employable amino acids derive from the group glycine, alanine, valine, leucin, isoleucin, phenylalanine, tyrosine, tryptophan, proline, aspartic acid, glutamic acid, asparagine, glutamine, serine, threonine, cystein, methionine, lysine, arginine, histidine, β-alanine, 4-amino butyric acid (GABA), betaine, L-cystine (L-Cyss), L-carnitine, L-citrulline, L-theanine, 3′,4′-dihydroxy-L-phenylalanine (L-Dopa), 5′-hydroxy-L-tryptophan, L-homocystein, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidin, and L-ornithine, wherein both individual amino acids as well as mixtures can be employed.

Preferred agents according to the invention comprise one or more amino acids in narrow quantitative ranges. Here, inventively preferred hair treatment agents comprise, based on total weight, 0.01 to 5 wt. %, preferably 0.02 to 2.5 wt. %, particularly preferably 0.05 to 1.5 wt. % more preferably 0.075 to 1 wt. % and particularly 0.1 to 0.25 wt. % of amino acid(s) as the care substance, preferably from the group of glycine and/or alanine and/or valine and/or lysine and/or leucine and/or threonine.

Vitamins, provitamins or vitamin precursors are a further preferred group of ingredients of the inventive agents. These are described below.

Retinol (vitamin A₁) as well as 3,4-didehydroretinol (vitamin A₂) belong in the group of substances designated as vitamin A. β-carotene is the provitamin of retinol. Examples of suitable vitamin A components according to the invention include vitamin A acid and its esters, vitamin A aldehyde and vitamin A alcohol, as well as its esters such as palmitate and acetate. Compositions according to the invention preferably comprise vitamin A components in amounts of 0.05 to 1 wt. %, based on total weight of the preparation.

The vitamin B group or vitamin B complex includes, inter alia—
Vitamin B₁ (Thiamine),
Vitamin B₂ (Riboflavin),
Vitamin B₃. The compounds nicotinic acid and nicotinamide (niacinamide) are often included under this designation. According to the invention, nicotinamide is preferred and is comprised in compositions according to the invention in amounts of 0.05 to 1 wt. % based on total weight of the composition.
Vitamin B₅ (pantothenic acid, panthenol and pantolactone). In the context of this group, panthenol and/or pantolactone is preferably used. Useable derivatives of panthenol according to the invention are especially the esters and ethers of panthenol as well as cationically derivatized panthenols. Specific representatives are for example, panthenol triacetate, panthenol monoethyl ether and its monoacetate as well as the cationic panthenol derivatives disclosed in WO 92/13829. The cited compounds of the vitamin B₅ type are preferably comprised in the compositions according to the invention in amounts of 0.05 to 10 wt. %, based on total weight of the composition. Quantities of 0.1 to 5 wt. % are particularly preferred.
Vitamin B₆ (pyridoxine as well as pyridoxamine and pyridoxal).
Vitamin C (ascorbic acid). Vitamin C is preferably added to the compositions according to the invention in amounts of 0.1 to 3 wt. %, based on total weight of the composition. Its use in the form of the palmitate ester the glucosides or phosphates can be preferred. The use in combination with tocopherols can also be preferred.
Vitamin E (Tocopherols, especially α-tocopherol). Tocopherol and its derivatives, particularly esters such as acetate, nicotinate, phosphate and succinate, are used in the compositions according to the invention preferably comprised in amounts of 0.05 to 1 wt. %, based on total weight of the composition.
Vitamin F. The term “vitamin F” typically refers to essential fatty acids, particularly linoleic acid, linolenic acid and arachidonoic acid.
Vitamin H. The compound (3aS,4S,6aR)-2-oxohexahydrothienol[3,4-d]-imidazole-4-valeric acid denotes Vitamin H, for which the common name biotin has become accepted. Compositions according to the invention preferably comprise biotin in amounts of 0.0001 to 1.0 wt. %, particularly in amounts of 0.001 to 0.01 wt. %.

In summary, inventively preferred hair treatment agents additionally comprise as care substances, based on total weight, 0.1 to 5 wt. %, preferably 0.2 to 4 wt. %, particularly preferably 0.25 to 3.5 wt. % more preferably 0.5 to 3 wt. % and particularly 0.5 to 2.5 wt. % of vitamins and/or provitamins and/or vitamin precursors, preferably attributed to the groups A, B, C, E, F and H, wherein preferred agents comprise panthenol ((±)-2,4-dihydroxy-N-(3-hydroxypropyl)-3,3-dimethylbutyramide, Provitamin B₅) and/or pantothenic acid (Vitamin B₃, Vitamin B₅) and/or niacin, niacinamide or nicotinamide (Vitamin B₃) and/or L-ascorbic acid (Vitamin C) and/or thiamine (Vitamin B₁) and/or riboflavin (Vitamin B₂, Vitamin G) and/or biotin (Vitamin B₇, Vitamin and/or folic acid (Vitamin B₉, Vitamin B₆ or Vitamin M) and/or Vitamin B₆ and/or Vitamin B₁₂.

It has been shown that the addition of certain quinones enhances an anti-dandruff effect and prevents hair loss as well as bringing advantages in regard to combability and shine. The inventive agents can therefore comprise 0.0001 to 5 wt. % of at least one bioquinone according to formula (I) as an additional ingredient

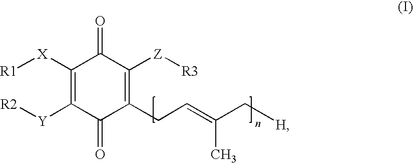

wherein X, Y, Z independently represent —O— or —NH— or NR⁴— or a chemical bond;
R¹, R², R³ independently represent hydrogen or an optionally substituted aryl group or an optionally substituted (C₁-C₆) alkyl group or a hydroxyalkyl group or a polyhydroxyalkyl group or an optionally substituted (C₁-C₆) alkylene group, or a (C₁-C₆) acyl group, wherein preferred groups independently include —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, and —C(CH₃)₃;
R⁴ stands for —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃; and
n represents values from 1 to 20, preferably from 2 to 15 and in particular from 5 to 10.

Inventively preferred compounds of Formula (I) include, for example—

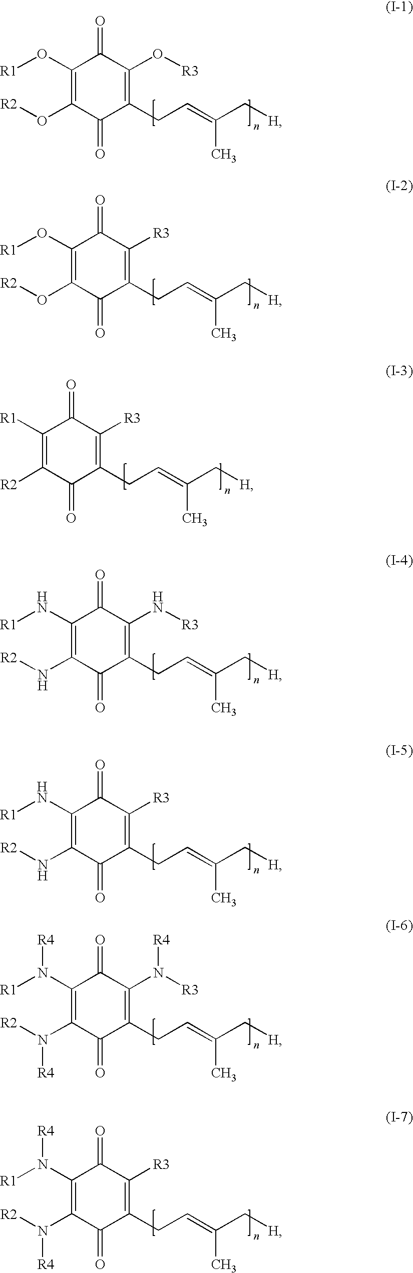

wherein R¹, R², R³ independently represents —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃;
R⁴ represents —CH₃, or —CH₂CH₃, or —(CH₂)₂CH₂, or —CH(CH₃)₂; and
n represents values from 1 to 20, preferably from 2 to 15 and in particular from 5 to 10.

According to the invention, particularly preferred hair treatment agents comprise, based on total weight of the agent, 0.0001 to 1 wt. %, preferably 0.001 to 0.5 wt. % and particularly preferably 0.005 to 0.1 wt. % of at least one ubiquinone and/or at least one ubiquinol and/or at least one derivative of these substances as a care substance, wherein preferred agents comprise an ubiquinone of the formula (Ubi)—

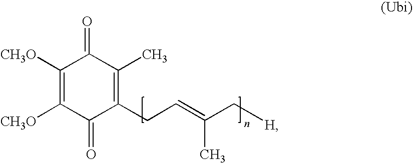

wherein n represents the values=6, 7, 8, 9 or 10, particularly preferably 10 (Coenzyme Q10).

The inventive agents can also comprise plastoquinones as an alternative to or in addition to the particularly preferred ubiquinones. Here, preferred agents according to the invention comprise 0.0002 to 4 wt. %, preferably 0.0005 to 3 wt. %, particularly preferably 0.001 to 2 wt. %, more preferably 0.0015 to 1, and especially 0.002 to 0.5 wt. % of at least one plastoquinone of the formula (Ib)—

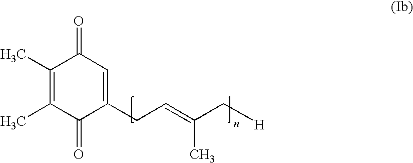

wherein n represents values from 1 to 20, preferably from 2 to 15 and especially from 5 to 10, wherein particularly preferred agents comprise plastoquinone PQ-9 of the formula

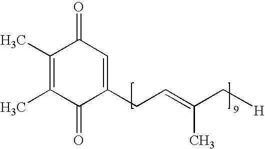

In order to improve elasticity and consolidation of the internal structure of hair treated with inventive agents, the inventive agents can include purine and/or purine derivatives. In particular, the combination of purine and/or purine derivatives with ubiquinones and/or plastoquinones means that hair treated with corresponding agents demonstrates, inter alia, higher measured values in differential thermo analysis and improved wet and dry combability.

Accordingly, the inventive agents can include purine and/or derivative(s) of purine as additional ingredients. Purine (7H-imidazo[4,5-d]pyrimidine) is not a naturally occurring product, but forms the parent substance of purines. Purines themselves are a group of important compounds that are commonly found in nature and participate in human, animal, vegetal and microbial metabolic processes, and are derived from the parent substance by substitution with OH, NH₂, SH in the 2-, 6- and 8-position and/or with CH₃ in the 1-, 3-, 7-position. Purine can be synthesized from amino acetonitrile and formamide, for example. Purine and/or purine derivatives are often isolated from natural products, but are also synthetically obtainable by numerous routes.

Preferred agents according to the invention comprise purine and/or purine derivatives in narrow quantitative ranges. Inventively preferred cosmetics comprise, based on their weight, 0.001 to 2.5 wt. %, preferably 0.0025 to 1 wt. %, particularly preferably 0.005 to 0.5 wt. % and particularly 0.01 to 0.1 wt. % purine and/or purine derivative(s).

According to the invention, some representatives are particularly preferred among purine, the purines and the purine derivatives. According to the invention, particularly preferred hair treatment agents comprise, based on their weight, 0.001 to 2.5 wt. %, preferably 0.0025 to 1 wt. %, particularly preferably 0.005 to 0.5 wt. % and in particular 0.01 to 0.1 wt. % of purine(s) and/or purine derivative(s) as a care substance, wherein preferred agents comprise purine(s) and/or purine derivative(s) of the formula (Pur-I)—

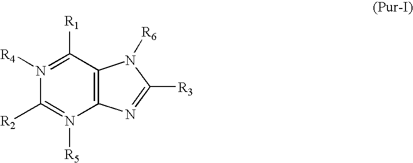

wherein the groups R¹, R² and R³ independently are —H, —OH, NH₂, or —SH; and the groups R⁴, R⁵ and R⁶ independently are —H, —CH₃ or —CH₂—CH₃, wherein the following substances are preferred:
Purine (R¹═R²═R³═R⁴═R⁵═R⁶═H),
Adenine (R¹═NH₂, R²═R³═R⁴═R⁵═R⁶═H),
Guanine (R¹═OH, R²═NH₂, R³═R⁴═R⁵═R⁶═H),
Uric acid (R¹═R²═R³═OH, R⁴═R⁵═R⁶═H),
Hypoxanthine (R¹═OH, R²═R³═R⁴═R⁵═R⁶═H),
6-Purine thiol (R¹═SH, R²═R³═R⁴═R⁵═R⁶═H),
6-Thioguanine (R¹═SH, R²═NH₂, R³═R⁴═R⁵═R⁶═H),
Xanthine (R¹═R²═OH, R³═R⁴═R⁵═R⁶═H),
Caffeine (R¹═R²═OH, R³═H, R⁴═R⁵═R⁶═CH₃),
Theobromine (R¹═R²═OH, R³═R⁴═H, R⁵═R⁶═CH₃), and
Theophylline (R¹═R²═OH, R³═H, R⁴═CH₃, R⁵═CH₃, R⁶═H).

The type and amount of the purine derivative can vary depending on the desired purpose of the inventive agent. Caffeine has proven effective especially in hair cosmetic formulations and can be used, for example, in shampoos in amounts of 0.005 to 0.25 wt. %, preferably 0.01 to 0.1 wt. % and especially 0.01 to 0.05 wt. % (each based on total weight of the shampoo).

It is further advantageous to employ purine or purine derivatives and bioquinone in a specific ratio to one another. Here, agents according to the invention are preferred in which the weight ratio of the ingredients a) and b) is 10:1 to 1:100, preferably 5:1 to 1:50, particularly preferably 2:1 to 1:20 g/l and especially 1:1 to 1:10.

As already mentioned, caffeine is a particularly preferred purine derivative and the coenzyme Q10 is a particularly preferred bioquinone. Accordingly, particularly preferred inventive agents comprise, based on total weight of the agent, 0.001 to 2.5 wt. %, preferably 0.0025 to 1 wt. %, particularly preferably 0.005 to 0.5 wt. % and especially 0.01 to 0.1 wt. % caffeine, and 0.0002 to 4 wt. %, preferably 0.0005 to 3 wt. %, particularly preferably 0.001 to 2 wt. %, more preferably 0.0015 to 1 and especially 0.002 to 0.5 wt. % coenzyme Q10.

The inventive agents can include at least one carbohydrate from the group of monosaccharides, disaccharides and/or oligosaccharides as a further ingredient. Here, inventively preferred hair treatment agents comprise, based on total weight of the agent, 0.01 to 5 wt. %, preferably 0.05 to 4.5 wt. %, particularly preferably 0.1 to 4 wt. % more preferably 0.5 to 3.5 wt. % and particularly 0.75 to 2.5 wt. % of carbohydrate(s) as the care substance, chosen from monosaccharides, disaccharides and/or oligosaccharides, wherein preferred carbohydrates are chosen from—

monosaccharides, in particular
D-Ribose and/or
D-Xylose and/or
L-Arabinose and/or
D-Glucose and/or
D-Mannose and/or
D-Galactose and/or
D-Fructose and/or
Sorbose and/or
L-Fucose and/or
L-Rhamnose, and

disaccharides, in particular
Saccharose and/or
Maltose and/or
Lactose and/or
Trehalose and/or
Cellobiose and/or
Gentiobiose and/or
Isomaltose.

Particularly preferred inventive agents comprise, based on total weight of the agent—

a) 0.005 to 0.015 wt. % caffeine and 0.75 to 1.5 wt. % glucose monohydrate,

b) 0.005 to 0.015 wt. % caffeine and 0.75 to 1.5 wt. % saccharose, and/or

c) 0.005 to 0.015 wt. % caffeine and 0.75 to 1.5 wt. % fructose

As already mentioned, preferred inventive agents comprise (an) amino acid(s). Inventively particularly preferred employable amino acids derive from the group glycine, alanine, valine, leucin, isoleucin, phenylalanine, tyrosine, tryptophan, proline, aspartic acid, glutamic acid, asparagine, glutamine, serine, threonine, cystein, methionine, lysine, arginine, histidine, β-alanine, 4-amino butyric acid (GABA), betaine, L-cystine (L-Cyss), L-carnitine, L-citrulline, L-theanine, 3′,4′-dihydroxy-L-phenylalanine (L-Dopa), 5′-hydroxy-L-tryptophan, L-homocystein, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidin, and L-ornithine, wherein both individual amino acids as well as mixtures can be employed.

Preferred agents according to the invention comprise one or more amino acids in narrow quantitative ranges. Here, inventively preferred cosmetic agents additionally comprise 0.05 to 5 wt. %, preferably 0.1 to 2.5 wt. %, particularly preferably 0.15 to 1 wt. % and particularly 0.2 to 0.5 wt % of amino acid(s), preferably (an) amino acid from the group of glycine and/or alanine and/or valine and/or lysine and/or leucine and/or threonine.

Particularly preferred inventive agents comprise, based on total weight of the agent—
a) 0.005 to 0.015 wt. % caffeine and 0.75 to 1.5 wt. % glucose monohydrate and 0.1 to 0.25 wt. % glycine,
b) 0.005 to 0.015 wt. % caffeine and 0.75 to 1.5 wt. % saccharose and 0.1 to 0.25 wt. % glycine,
c) 0.005 to 0.015 wt. % caffeine and 0.75 to 1.5 wt. % fructose and 0.1 to 0.25 wt. % glycine,
d) 0.005 to 0.015 wt. % caffeine and 0.75 to 1.5 wt. % glucose monohydrate and 0.1 to 0.25 wt. % alanine,
e) 0.005 to 0.015 wt. % caffeine and 0.75 to 1.5 wt. % saccharose and 0.1 to 0.25 wt. % alanine,
f) 0.005 to 0.015 wt. % caffeine and 0.75 to 1.5 wt. % fructose and 0.1 to 0.25 wt. % alanine,
g) 0.005 to 0.015 wt. % caffeine and 0.75 to 1.5 wt. % glucose monohydrate and 0.1 to 0.25 wt. % valine,
h) 0.005 to 0.015 wt. % caffeine and 0.75 to 1.5 wt. % saccharose and 0.1 to 0.25 wt. % valine, and/or
i) 0.005 to 0.015 wt. % caffeine and 0.75 to 1.5 wt. % fructose and 0.1 to 0.25 wt. % valine.

According to the invention, preferred agents comprise, based on their weight, 0.01 to 15 wt. %, preferably 0.025 to 12.5 wt. %, particularly preferably 0.05 to 10 wt. %, more preferably 0.1 to 7.5 wt. % and particularly 0.5 to 5 wt. % of at least one furanone derivative of the formula (Fur-I) and/or of the formula (Fur-II) as the care substance—

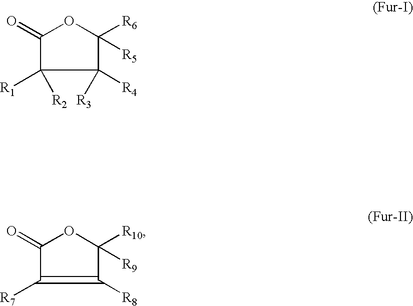

wherein the groups R¹ to R¹⁰ independently represent—
hydrogen, —OH, a methyl, methoxy, aminomethyl or hydroxymethyl group,
C₂-C₄ saturated or mono or di-unsaturated, branched or linear hydrocarbon groups,
C₂-C₄ saturated or mono or di-unsaturated, branched or linear mono-, di- or trihydroxyhydrocarbon groups,
C₂-C₄ saturated or mono or di-unsaturated, branched or linear mono-, di- or triamino hydrocarbon groups,
an —OR¹¹ group, with —R¹¹ as a —C₂-C₄ saturated or mono or di-unsaturated, branched or linear hydrocarbon group, —C₂-C₄ saturated or mono or di-unsaturated, branched or linear mono-, di- or trihydroxyhydrocarbon group,
an —NR¹²R¹³ group, wherein R¹² and R¹³ each independently of one another stand for hydrogen, a methyl, a —C₂-C₄ saturated or mono- or di-unsaturated, branched or linear hydrocarbon group, a C₂-C₄ saturated or mono- or di-unsaturated, branched or linear mono-, di- or trihydroxyhydrocarbon group,
a —COOR¹⁴ group, wherein R¹⁴ stands for hydrogen, a methyl, a —C₂-C₄ saturated or mono- or di-unsaturated, branched or linear hydrocarbon group, a —C₂-C₄ saturated or mono- or di-unsaturated, branched or linear mono-C₂-C₄ di- or trihydroxyhydrocarbon group, a C₂-C₄ saturated or mono or di-unsaturated, branched or linear mono-, di- or triamino hydrocarbon group,
a —CONR¹⁵R¹⁶ group, wherein R¹⁵ and R¹⁶ each stand for hydrogen, methyl, a —C₂-C₄ saturated or mono- or di-unsaturated, branched or linear hydrocarbon group, a —C₂-C₄ saturated mono- or di-unsaturated, branched or linear mono-, di- or trihydroxyhydrocarbon group, a C₂-C₄ saturated or mono- or di-unsaturated, branched or linear mono-, di- or triamino hydrocarbon group,
a —COR¹⁶ group, wherein R¹⁶ stands for hydrogen, a methyl, a —C₂-C₄ saturated or mono- or di-unsaturated, branched or linear hydrocarbon group, a —C₂-C₄ saturated or mono- or di-unsaturated, branched or linear mono- di- or trihydroxyhydrocarbon group, a C₂-C₄ saturated or mono or di-unsaturated, branched or linear mono-, di- or triamino hydrocarbon group,
an —OCOR¹⁷ group, wherein R¹⁷ stands for a methyl, a —C₂-C₃₀ saturated or mono- or polyunsaturated, branched or linear hydrocarbon group, a —C₂-C₃₀ saturated or mono- or polyunsaturated, branched or linear mono-, di-, tri- or polyhydroxyhydrocarbon group, a C₂-C₃₀ saturated or mono or poly-unsaturated, branched or linear mono-, di-, tri- or polyamino hydrocarbon group,
   with the proviso that for the case where R⁷ and R⁸ represent —OH and simultaneously R⁹ or R¹⁰ represents hydrogen, then the remaining group R⁹ or R¹⁰ does not represent a dihydroxyethyl group.

Compounds of the formulas (Fur-I) and (Fur-II) are employed as intermediates in the synthesis of natural products and in the manufacture of pharmaceuticals and vitamins. The active substances according to the formulas (Fur-I) and (Fur-II) can be manufactured for example by treating primary alcohols with acrylic acids. In addition, compounds of Formula (Fur-I) can be obtained by reactions starting from hydroxypivaldehyde. Likewise, alkynes can be carbonylated to form substituted 2-furanones of the formula (Fur-I) or (Fur-II). Finally, the compounds of formula (Fur-I) or of formula (Fur-II) can be obtained by intramolecular esterification of the corresponding hydroxycarboxylic acids. For example, the following compounds are obtained by one of the listed synthetic routes:

2,5-dihydro-5-methoxy-2-furanone, tetrahydro-5-oxo-2-furan carboxylic acid, dihydro-3-hydroxy-4,4-dimethyl-2(3H)-furanone, or 3,4-dimethyl-5-pentylidenedihydro-2(5H)-furanone or 4-hydroxy-2,5-dimethyl-3(2H)-furanone. Naturally, the 2-furanones according to the invention include all possible stereoisomers as well as their mixtures. 2-furanones according to the invention do not sustainably influence the odor of the cosmetic agent, such that the agent has to be separately perfumed.

Preferred compounds of the formula (Fur-I) and/or of the formula (Fur-II) can be compounds, in which the substituents R¹, R² and R⁷ independently represent:
a) hydrogen, an —OH, a methyl, methoxy, aminomethyl, hydroxymethyl group,
b) a —C₂-C₄ saturated or mono or di-unsaturated, branched or linear mono-, di- or trihydroxyhydrocarbon group,
c) a —C₂-C₄ saturated or mono or di-unsaturated, branched or linear mono-, di- or triamino hydrocarbon group,
d) an —OR¹¹ group, with —R¹¹ as a —C₂-C₄ saturated or mono or di-unsaturated, branched or linear hydrocarbon group, —C₂-C₄ saturated or mono or di-unsaturated, branched or linear mono-, di- or trihydroxyhydrocarbon groups,
e) an —NR¹²R¹³ group, wherein R¹² and R¹³ each independently of one another stand for hydrogen, a methyl, a —C₂-C₄ saturated or mono- or di-unsaturated, branched or linear hydrocarbon group, a —C₂-C₄-gesättigten saturated or mono- or di-unsaturated, branched or linear mono-, di- or trihydroxyhydrocarbon group,
f) a —COOR¹⁴ group, wherein R¹⁴ stands for hydrogen, a methyl, a —C₂-C₄ saturated or mono- or di-unsaturated, branched or linear hydrocarbon group, a —C₂-C₄ saturated or mono- or di-unsaturated, branched or linear mono- di- or trihydroxyhydrocarbon group, a C₂-C₄ saturated or mono or di-unsaturated, branched or linear mono-, di- or triamino hydrocarbon group,
g) a —COR¹⁶ group, wherein R¹⁶ stands for hydrogen, a methyl, a —C₂-C₄ saturated or mono- or di-unsaturated, branched or linear hydrocarbon group, a —C₂-C₄ saturated or mono- or di-unsaturated, branched or linear mono- di- or trihydroxyhydrocarbon group, a C₂-C₄ saturated or mono or di-unsaturated, branched or linear mono-, di- or triamino hydrocarbon group,

h) an —OCOR¹⁷ group, wherein R¹⁷ stands for a methyl, a —C₂-C₃₀ saturated or mono- or di-unsaturated, branched or linear hydrocarbon group, a —C₂-C₃₀ saturated or mono- or polyunsaturated, branched or linear mono-, di-, tri- or polyhydroxyalkyl group, a —C₂-C₃₀ saturated or mono or poly-unsaturated, branched or linear mono-, di-, tri- or polyamino hydrocarbon group.

In a further embodiment of the inventive teaching, the groups R³, R⁴ and R⁸, independently in the compounds of formula (Fur-I) or of formula (Fur-II) preferably represent:
hydrogen, an —OH, a methyl, methoxy, aminomethyl, hydroxyethyl group,
a —C₂-C₄ saturated or mono or di-unsaturated, branched or linear hydrocarbon group,
a —C₂-C₄ saturated or mono or di-unsaturated, branched or linear mono-, di- or trihydroxyhydrocarbon group, or
a —C₂-C₄ saturated or mono or di-unsaturated, branched or linear mono-, di- or triamino hydrocarbon group

In addition, in the inventive active substance according to formula (I) and/or Formula (II), it can be preferred if the groups R⁵, R⁶, R⁹ and R¹⁰ independently represent:
hydrogen, an —OH, a methyl, methoxy, aminomethyl, hydroxymethyl group,
a —C₂-C₄ saturated or mono or di-unsaturated, branched or linear hydrocarbon group,
a —C₂-C₄ saturated or mono or di-unsaturated, branched or linear mono-, di- or trihydroxyhydrocarbon group, or
a) a —C₂-C₄ saturated or mono or di-unsaturated, branched or linear mono-, di- or triamino hydrocarbon group.

In a particularly preferred embodiment of the inventive teaching, a compound of formula (Fur-I) is employed. It can be preferred if in a compound of formula (Fur-I), the groups R¹ and R² independently represent:
a) hydrogen, an —OH, a methyl, methoxy, aminomethyl, hydroxymethyl group,
b) a —C₂-C₄ saturated or mono or di-unsaturated, branched or linear mono-, di- or trihydroxyhydrocarbon group,
c) an —OR¹¹ group, with R¹¹ as a —C₂-C₄-saturated or mono or di-unsaturated, branched or linear hydrocarbon group,
d)-C₂-C₄ saturated or mono or di-unsaturated, branched or linear mono-, di- or trihydroxyhydrocarbon groups,
e) a —COOR¹⁴ group, wherein R¹⁴ stands for hydrogen, a methyl, a —C₂-C₄ saturated or mono- or di-unsaturated, branched or linear hydrocarbon group, a —C₂-C₄ saturated mono- or di-unsaturated, branched or linear mono-, di- or trihydroxyhydrocarbon group,
f) a —COR¹⁶ group, wherein R¹⁶ stands for a methyl, a —C₂-C₄ saturated or mono- or di-unsaturated, branched or linear hydrocarbon group, a —C₂-C₄ saturated or mono- or di-unsaturated, branched or linear mono-, di- or trihydroxyhydrocarbon group, or
g) an —OCOR¹⁷ group, wherein R¹⁷ stands for a methyl, a —C₂-C₃₀ saturated or mono- or polyunsaturated, branched or linear hydrocarbon group, a —C₂-C₃₀ saturated or mono- or polyunsaturated, branched or linear mono-, di-, tri- or polyhydroxyhydrocarbon group

In addition, in this particularly preferred embodiment of the inventive teaching, it can be advantageous if in the compounds of formula (Fur-I), the groups R³ and R⁴ independently represent:
a) hydrogen, an —OH, a methyl, methoxy, aminomethyl, hydroxymethyl group,
b) a —C₂-C₄ saturated or mono or di-unsaturated, branched or linear mono-, di- or trihydroxyhydrocarbon group,
c) an —OR¹¹ group, with —R¹¹ as a —C₂-C₄ saturated or mono or di-unsaturated, branched or linear hydrocarbon group, —C₂-C₄ saturated or mono or di-unsaturated, branched or linear mono-, di- or trihydroxyhydrocarbon groups,
d) a —COOR¹⁴ group, wherein R¹⁴ stands for hydrogen, a methyl, a —C₂-C₄ saturated or mono- or di-unsaturated, branched or linear hydrocarbon group, a —C₂-C₄ saturated mono- or di-unsaturated, branched or linear mono-, di- or trihydroxyhydrocarbon group,
e) a —OCOR¹⁷ group, wherein R¹⁷ stands for a methyl, a —C₂-C₃₀ saturated or mono- or polyunsaturated, branched or linear hydrocarbon group, a —C₂-C₃₀ saturated or mono- or polyunsaturated, branched or linear mono-, di- tri- and/or polyhydroxyhydrocarbon group.

In this preferred embodiment, it can be further advantageous if in the compounds of formula (Fur-I), the groups R⁵ and R⁶ independently represent:
a) a —C₂-C₄ saturated or mono or di-unsaturated, branched or linear mono-, di- or trihydroxyhydrocarbon group, or
b) an —OR¹¹ group, with —R¹¹ as a —C₂-C₄ saturated or mono or di-unsaturated, branched or linear hydrocarbon group, —C₂-C₄ saturated or mono or di-unsaturated, branched or linear mono-, di- or trihydroxyhydrocarbon group.

In a particularly preferred embodiment of the inventive teaching, the added compound that corresponds to formula (Fur-I) is—
a) (R)-(+4-hydroxymethyl-γ-butyrolactone and/or
b) D,L-4-hydroxymethyl-γ-butyrolactone and/or
c) (S)-(+)-4-hydroxymethyl-γ-butyrolactone and/or
d) R-(−)-2-hydroxy-3,3-dimethyl-γ-butyrolactone and/or
e) D,L-2-hydroxy-3,3-dimethyl-γ-butyrolactone and/or
f) S(+)-2-hydroxy-3,3-dimethyl-γ-butyrolactone and/or
g) 4-hydroxy-2,5-dimethyl-3(2H)-furanone and/or
h) tetrahydro-5-oxo-2-furancarboxylic acid and/or
i) tetrahydro-5-oxo-2-furancarboxylic acid, Na-salt and/or
j) tetrahydro-5-oxo-2-furancarboxylic acid, K-salt and/or
k) 2,5-dihydro-5-methoxy-2-furanone and/or
l) dihydro-3-hydroxy-4,4-dimethyl-2(3H)-furanone.

In a quite particularly preferred embodiment of the inventive teaching, dihydro-3-hydroxy-4,4-dimethyl-2(3H)-furanone is employed as the compound corresponding to Formula (Fur-I).

A further preferred employable care substance that possesses activating properties is taurine. Inventively preferred hair treatment agents comprise, based on total weight of the agent, 0.01 to 15 wt. %, preferably 0.025 to 12.5 wt. %, particularly preferably 0.05 to 10 wt. %, more preferably 0.1 to 7.5 wt. % and particularly 0.5 to 5 wt. % of taurine (2-aminoethane sulfonic acid) as a care substance.

The additional use of bisabolol and/or bisabolol oxides is also preferred in the inventive agents. Here, hair treatment agents according to the invention are preferred, which additionally comprise 0.001 to 5 wt. %, preferably 0.01 to 4 wt. %, particularly preferably 0.02 to 2.5 wt. % and especially 0.1 to 1.5 wt. % bisabolol and/or oxides of bisabolol, preferably (−)-alpha-bisabolol

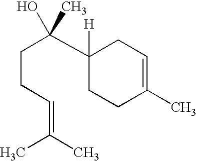

In addition to the chitosan (derivative)(s), the silicone elastomers and optional further ingredients, the inventive agents can comprise additional substances that prevent, mitigate or heal hair loss. A content of hair root-stabilising active substances is particularly advantageous. These substances will be described below.

At present Propecia (Finasterid) is the sole preparation that is approved world wide and for which an efficacy and tolerance has been proven in numerous studies. Propecia acts by reducing the amount of DHT formed from testosterone.

Minoxidil, with or without supplementary additives, is probably the oldest demonstrably effective hair growth agent. It may only be used externally to treat hair loss. There exist hair tonics which comprise 2%-5% Minoxidil, and gels with up to 15% Minoxidil. The efficacy increases with the dose, although Minoxidil is only soluble to 5% in hair tonics. Hair tonics with up to 3% Minoxidil content are obtainable without prescription in many countries.

Spironolactone in the form of hair tonic and in combination with Minoxidil can be used for external application in order to combat the hormonal effects on the hair follicle. Spironolactone acts as an androgen receptor blocker, meaning, the binding of DHT onto the hair follicle is impeded.

In summary, preferred cosmetic agents according to the invention additionally comprise, based on total weight of the agent, 0.001 to 5 wt. % hair root-stabilizing substance, especially Minoxidil and/or Finasterid and/or Ketoconazol.

Additional anti-dandruff active substances (e.g., Climbazol, Piroctone Olamine or Zinc-Pyrithion) specifically reduce the amount of yeast that causes dandruff, the bacterial flora again attains the normal percentage composition and the desquamation is reduced to the physiological level. However, laboratory tests have shown that different varieties of the Pityrosporum ovale react with varying degrees of success to the anti-dandruff active substances. In order to best combat all dandruff pathogens, a combination of anti-dandruff active substances is therefore the most successful.

In summary, preferred inventive hair treatment agents additionally comprise, based on total weight of the agent, 0.001 to 5 wt. % anti-dandruff active substances, especially Piroctone Olamine (1-hydroxy-4-methyl-6-(2,4,4-trimethylpentyl)pyridin-2(1H)-one, compound with 2-aminoethanol, 1:1) and/or zinc-pyrithion and/or selenium sulfide and/or Climbazol and/or salicylic acid or fumaric acid.

The inventive agents can furthermore comprise all active substances, additives and auxiliaries known for such preparations. In many cases the compositions comprise at least one surfactant, wherein, in principle, not only anionic, but also zwitterionic, ampholytic, non-ionic and cationic surfactants are suitable. However, in many cases it has proved advantageous to select surfactants from among anionic, zwitterionic or non-ionic surfactants. These surfactants were described in detail above.

In a further preferred embodiment, the inventive compositions can comprise emulsifiers (F). Emulsifiers act at the oil/water interface to produce water or oil-stable adsorption layers that protect the dispersed droplets against coalescence and thereby stabilize the emulsion. Thus, emulsifiers, like surfactants are composed of hydrophobic and hydrophilic molecular moieties. Hydrophilic emulsifiers preferably form O/W emulsions and hydrophobic emulsifiers preferably form W/O emulsions. An emulsion is understood to mean a dispersion of a liquid in the form of droplets in another liquid using an energy input to afford interfaces stabilized with surfactants. The choice of this emulsifying surfactant or emulsifier depends on the materials being dispersed and the respective external phase as well as the fineness of the emulsion. Exemplary emulsifiers usable according to the invention are
a) Addition products of 4 to 30 moles ethylene oxide and/or 0 to 5 moles propylene oxide to linear fatty alcohols containing 8 to 22 carbon atoms, to fatty acids containing 12 to 22 carbon atoms and to alkyl phenols containing 8 to 15 carbon atoms in the alkyl group,
b) C₁₂-C₂₂ fatty acid mono- and diesters of addition products of 1 to 30 moles ethylene oxide on polyols containing 3 to 6 carbon atoms, especially glycerine,
c) ethylene oxide and polyglycerine addition products on methyl glucoside fatty acid esters, fatty acid alkanolamides and fatty acid glucamides,
d) C₈-C₂₂ alkyl mono and oligoglycosides and their ethoxylated analogs, wherein the degrees of oligomerization are 1.1 to 5, particularly 1.2 to 2.0, and glucose as the sugar component are preferred,
e) mixtures of alkyl(oligo) glucosides and fatty alcohols, for example the commercial product Montanov® 68,
f) addition products of 5 to 60 mole ethylene oxide onto castor oil and hydrogenated castor oil, partial esters of polyols containing 3-6 carbon atoms with saturated fatty acids containing 8 to 22 carbon atoms,
g) sterols. Sterols are understood to mean a group of steroids, which carry a hydroxyl group on carbon atom 3 of the steroid skeleton and are isolated from both animal tissue (zoosterols) and vegetal fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.
h) phospholipids. Among these are principally meant the glucose-phospholipids, which are obtained e.g. as lecithins or phosphatidyl cholines from, for example, egg yolk or plant seeds (e.g., soya beans).
i) fatty acid esters of sugars and sugar alcohols such as sorbitol,

j) polyglycerines and polyglycerine derivatives such as for example polyglycerine poly-12-hydroxystearate (commercial product Dehymuls® PGPH), and
k) linear and branched fatty acids with 8 to 30 carbon atoms and their Na, K, ammonium, Ca, Mg and Zn salts.

The inventive compositions preferably comprise the emulsifiers in quantities of 0.1 to 25 wt. %, particularly 0.5-15 wt. %, based on the total composition.

Preferably, the inventive compositions can comprise at least one non-ionic emulsifier with an HLB value of 8 to 18. Non-ionic emulsifiers with an HLB value of 10-15 can be particularly preferred according to the invention.

It has also been shown to be advantageous if, in addition to polymer(s) from the group of cationic and/or amphoteric polymers, further polymers (G) are included in the inventive agents. In a preferred embodiment, further polymers are accordingly added to the inventively employed agents, wherein not only anionic but also non-ionic polymers have proven efficient.

Anionic polymers (G2) concern anionic polymers that possess carboxylate and/or sulfonate groups. Exemplary anionic monomers, from which such polymers can be made include acrylic acid, methacrylic acid, crotonic acid, maleic anhydride and 2-acrylamido-2-methylpropane sulfonic acid. Here, the acidic groups may be fully or partially present as sodium, potassium, ammonium, mono- or triethanolammonium salts. Preferred monomers include 2-acrylamido-2-methylpropane sulfonic acid and acrylic acid.

Anionic polymers that comprise 2-acrylamido-2-methylpropane sulfonic acid alone or as a comonomer have proven to be quite particularly effective. The sulfonic acid group may be fully or partially present as the sodium, potassium, ammonium, mono- or triethanolammonium salt.

The homopolymer of 2-acrylamido-2-methylpropane sulfonic acid, which is commercially available, for example, under the trade name Rheothik® 11-80, is particularly preferred.

In this embodiment, it can be preferred to use copolymers of at least one anionic monomer and at least one non-ionic monomer. Regarding the anionic monomers, reference is made to the abovementioned substances. Preferred non-ionic monomers include acrylamide, methacrylamide, acrylic acid esters, methacrylic acid esters, vinyl pyrrolidone, vinyl ethers and vinyl esters.

Preferred anionic copolymers include acrylic acid-acrylamide copolymers and particularly polyacrylamide copolymers with monomers containing sulfonic acid groups. A particularly preferred anionic copolymer consists of 70 to 55 mole % acrylamide and 30 to 45 mole % 2-acrylamido-2-methylpropane sulfonic acid, wherein the sulfonic acid group may be fully or partially present as the sodium, potassium, ammonium, mono- or triethanolammonium salt. This copolymer can also be crosslinked, wherein the preferred crosslinking agents include polyolefinically unsaturated compounds such as tetraallyloxyethane, allyl sucrose, allyl pentaerythritol and methylene bisacrylamide. Such a polymer is comprised in the commercial product Sepigel®305 from the SEPPIC Company. The use of this compound, which comprises a mixture of hydrocarbons (C₁₃-C₁₄ isoparaffins) and a non-ionic emulsifier (Laureth-7) in addition to the polymer components, has proved to be particularly advantageous in the context of the inventive teaching.

Sodium acryloyl dimethyl taurate copolymers commercialized as a compound with isohexadecane and polysorbate 80 under the trade name Simulgel®600 have also proved to be particularly effective according to the invention.

Likewise preferred anionic homopolymers are uncrosslinked and crosslinked polyacrylic acids. Here the preferred crosslinking agents can be allyl ethers of pentaerythritol, of sucrose and of propylene. Such compounds are commercially available under the trade name Carbopol®, for example.

Copolymers of maleic anhydride and methyl vinyl ether, especially those with crosslinks, are also color-conserving polymers. A maleic acid-methyl vinyl ether copolymer, crosslinked with 1,9-decadiene is commercially available under the name Stabileze® QM.

In a further variant, the inventive compositions can additionally comprise non-ionic polymers (G4).

Suitable non-ionic polymers include, for example:
a) vinyl pyrrolidone-vinyl ester copolymers, such as those marketed by BASF under the trade name Luviskol®, Luviskol® VA 64 and Luviskol® VA 73, each vinyl pyrrolidone-vinyl acetate copolymers, are likewise preferred non-ionic polymers;
b) cellulose ethers, such as hydroxypropyl cellulose, hydroxyethyl cellulose, and methyl hydroxypropyl cellulose, as marketed for example under the trademarks Culminal® and Benecel® (AQUALON) and Natrosol® types (Hercules);
c) starch and its derivatives, especially starch ethers, for example Structure® XL (National Starch), a multifunctional, salt tolerant starch;
d) shellac;
e) polyvinyl pyrrolidones, as are marketed, for example, under the designation Luviskol®(BASF);
siloxanes. These siloxanes can be both water-soluble and also water-insoluble. Both volatile and non-volatile siloxanes are suitable, whereby non-volatile siloxanes are understood to mean such compounds with a boiling point above 200° C. at normal pressure. Preferred siloxanes are polydialkylsiloxanes, such as, for example polydimethylsiloxane, polyalkylarylsiloxanes, such as, for example polyphenylmethylsiloxane, ethoxylated polydialkylsiloxanes, as well as polydialkylsiloxanes, which comprise amine and/or hydroxyl groups; and
g) glycosidically substituted silicones.

According to the invention, it is also possible for the preparations to comprise a plurality, particularly two different polymers of the same charge and/or each with an anionic and an amphoteric and/or non-ionic polymer.

Compositions according to the invention preferably comprise the polymers (G) in quantities of 0.05 to 10 wt. %, based on total composition. Quantities of 0.1 to 5 wt. %, particularly 0.1 to 3 wt. %, are particularly preferred.

Moreover, in a preferred embodiment of the invention, an inventive composition can also include UV filters (I). The inventively useable UV filters are not generally limited in regard to their structure and their physical properties. Indeed, all UV filters that can be employed in the cosmetic field having an absorption maximum in the UVA (315-400 nm), in the UVB (280-315 nm) or in the UVC (<280 nm) regions are suitable. UV filters having an absorption maximum in the UVB region, especially in the range from about 280 to about 300 nm, are particularly preferred.

UV-filters used in accordance with the invention include, for example, substituted benzophenones, p-aminobenzoates, diphenylacrylates, cinnamates, salicylates, benzimidazoles and o-aminobenzoates.

Exemplary inventively useable UV filters include 4-aminobenzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and its potassium, sodium and triethanolamine salts (Phenylbenzimidazole sulfonic acid; Parsol®HS; Neo Heliopan®Hydro), 3,3′-(1,4-phenylenedimethylene)-bis(7,7-dimethyl-2-oxo-bicyclo-[2.2.1]hept-1-yl-methane sulfonic acid) and its salts, 1-(4-tert.-butylphenyl)-3-(4-methoxyphenyl)-propane-1,3-dione (Butyl methoxydibenzoylmethane; Parsol®1789, Eusolex®9020), α-(2-oxoborn-3-ylidene)-toluene-4-sulfonic acid and its salts, ethoxylated 4-aminobenzoic acid ethyl ester (PEG-25 PABA; Uvinul®P 25), 4-dimethylaminobenzoic acid 2-ethylhexyl ester (Octyl Dimethyl PABA; Uvasorb®DMO, Escalol®507, Eusolex®6007), salicylic acid 2-ethylhexyl ester (Octyl Salicylat; Escalol®587, Neo Heliopan®OS, Uvinul®O18), 4-methoxycinnamic acid isopentyl ester (Isoamyl p-methoxycinnamate; Neo Heliopan®1000), 4-methoxycinnamic acid 2-ethylhexyl ester (Octyl Methoxycinnamate; Parsol®MCX, Escalol®557, Neo Heliopan®AV), 2-hydroxy-4-methoxybenzophenone-5-sulfonic acid and its sodium salt (Benzophenone-4; Uvinul®MS 40; Uvasorb®S 5), 3-(4′-methylbenzylidene)-D,L-camphor (4-Methylbenzylidene camphor; Parsol®5000, Eusolex®6300), 3-benzylidene camphor (3-Benzylidene camphor), 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N-{(2 and 4)-[2-oxoborn-3-ylidenemethyl]benzyl}-acrylamide, 2,4-dihydroxybenzophenone (Benzophenone-1; Uvasorb®20H, Uvinul®400), 1,1′-diphenylacrylonitrilic acid 2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid 2′-ethylhexyl ester. Preferred are 4-aminobenzoic acid, N,N,N-trimethyl-4-(2-oxoborn-3-ylidenemethyl)aniline methylsulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethylene)-bis(7,7-dimethyl-2-oxo-bicyclo-[2.2.1]hept-1-yl methanesulfonic acid) and its salts, 1-(4-tert.-butylphenyl)-3-(4-methoxyphenyl)-propane-1,3-dione, α-(2-oxoborn-3-ylidene)-toluene-4-sulfonic acid and its salts, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and its sodium salt, 3-(4′-methylbenzylidene)-D,L-camphor, 3-benzylidene-camphor, 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N-{(2 and 4)-[2-oxoborn-3-ylidenemethyl]benzyl}-acrylamide. According to the invention, 2-hydroxy-4-methoxy-benzophenone, 2-phenylbenzimidazole-5-sulfonic acid and its potassium, sodium and triethanolamine salts, 1-(4-tert.-butylphenyl)-3-(4-methoxyphenyl)-propane-1,3-dione, 4-methoxycinnamic acid 2-ethylhexyl ester and 3-(4′-methylbenzylidene)-D,L-camphor are quite particularly preferred.

UV filters having a molecular extinction coefficient at the absorption maximum of above 15,000, particularly 20,000, are preferred.

Moreover, it was found that for structurally similar UV filters, in many cases in the context of the inventive teaching, the water-insoluble compound exhibits a higher activity than that of water-soluble compounds that differ from them by one or a plurality of additional ionic groups. In the context of the invention, water-insoluble UV filters are understood to mean those that do not dissolve more than 1 wt. %, especially not more than 0.1 wt. % in water at 20° C. In addition, these compounds should be soluble to at least 0.1, especially to at least 1 wt. % in conventional cosmetic oil components at room temperature. Accordingly, the use of water-insoluble UV filters can be inventively preferred.

According to a further embodiment of the invention, those UV filters are preferred which have a cationic group, especially a quaternary ammonium group.

These UV filters have the generic structure U-Q.

Here, the structural component U stands for a group that absorbs UV radiation. In principle, these groups can derive from the known abovementioned UV filters that can be employed in the field of cosmetics, in which one group, generally a hydrogen atom of the UV filter is replaced by a cationic group Q, in particular with a quaternary amino function. Compounds, from which the structural component U can derive are for example—

a) substituted benzophenones,

b) p-aminobenzoic acid esters,

c) diphenylacrylic acid esters,

d) cinnamic acid esters,

e) salicylic acid esters,

f) benzimidazoles, and

g) o-aminobenzoic acid esters

Structural components U that derive from cinnamic acid amide or from N,N-dimethylaminobenzoic acid amide are inventively preferred.

In principle, the structural components U can be selected such that the absorption maximum of the UV filter can be in the UVA (315-400 nm) region, as well as in the UVB (280-315 nm) region or in the UVC (<280 nm) region. UV filters having an absorption maximum in the UVB region, especially in the range from about 280 to about 300 nm, are particularly preferred.

Furthermore, the structural component U, also depending on the structural component Q, is preferably selected such that the molar extinction coefficient of the UV filter at the absorption maximum is above 15,000, especially above 20,000.

The structural component Q preferably comprises a quaternary ammonium group as the cationic group. In principle, this quaternary ammonium group can be directly bonded to the structural component U such that the structural component U represents one of the four substituents of the positively charged nitrogen atom. Preferably however, one of the four substituents on the positively charged nitrogen atom is a group, in particular, an alkyl group containing 2 to 6 carbon atoms, which acts as the link between the structural component U and the positively charged nitrogen atom.

Advantageously, the group Q has the general structure —(CH₂)ₓ—N⁺R¹R²R³X⁻, wherein x represents an integer from 1 to 4; R¹ and R² independently represent C₁₋₄ alkyl groups; R³ represents a C₁₋₂₂ alkyl group or a benzyl group; and X⁻ is a physiologically compatible anion. In the context of this general structure, x preferably represents the number 3, R¹ and R² each a methyl group, and R³ either a methyl group or a saturated or unsaturated, linear or branched hydrocarbon chain containing 8 to 22, particularly 10 to 18 carbon atoms.

Exemplary physiologically compatible anions include inorganic anions such as halides, particularly chloride, bromide and fluoride, sulfate ions and phosphate ions, as well as organic anions such as lactate, citrate, acetate, tartrate, methosulfate and tosylate.

Two preferred UV filters containing cationic groups are the commercially available compounds cinnamic acid amidopropyltrimethylammonium chloride (Incroquat® UV-283) and dodecyldimethylaminobenzamidopropyldimethyl ammonium tosylate (Escalol® HP 610).

Of course, the inventive teaching also includes use of a combination of a plurality of UV filters. In the context of this embodiment, the combination of at least one water-insoluble UV filter with at least one UV filter containing a cationic group is preferred.

The compositions according to the invention preferably comprise the UV filters (I) in quantities of 0.1 to 5 wt. %, based on the total composition. Quantities of 0.4-2.5 wt. % are preferred.

The inventive compositions can further include a 2-pyrrolidinone-5-carboxylic acid and derivatives (J) thereof. The sodium, potassium, calcium, magnesium or ammonium salts are preferred, wherein the ammonium ion carries one to three C₁- to C₄ alkyl groups besides hydrogen. The sodium salt is quite particularly preferred. The quantities employed in the inventive compositions preferably range from 0.05 to 10 wt. %, based on total composition, particularly preferably 0.1 to 5, and particularly 0.1 to 3 wt. %.

Finally, the inventive composition can also comprise plant extracts (L). Usually, these extracts are manufactured by extraction of the whole plant. In individual cases, however, it can also be preferred to produce the extracts solely from blossoms and/or leaves of the plant.

With regard to the inventively usable plant extracts, we particularly refer to extracts that are listed in the Table beginning on page 44 of the 3rd edition of the Guidelines for the Declaration of Ingredients in Cosmetics (Leitfadens zur Inhaltsstoffdeklaration kosmetischer Mittel), published by the German Cosmetics, Toiletry, Perfumery and Detergent Association e.V. (IKW), Frankfurt.

According to the invention, mainly extracts from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred.

Extracts from green tea, oak bark, stinging nettle, hamamelis, hops, camomile, burdock root, hawthorn, linden flowers, almonds, aloe vera, coconut, mango, apricot, lime, wheat, kiwi, melon, orange, grapefruit, sage, rosemary, birch, lady's smock, common yarrow, rest-harrow, meristem, ginseng and ginger are preferred.

Extracts of green tea, almonds, aloe vera, coconut, mango, apricot, lime, wheat, kiwi and melon are quite particularly suitable for the inventive use.

The extraction composition used to prepare the cited plant extracts can be water, alcohols, as well as their mixtures. Exemplary preferred alcohols are lower alcohols such as ethanol and isopropanol, but particularly polyhydroxy alcohols such as ethylene glycol, propylene glycol and butylene glycol, both as the sole extracting composition as well as in aqueous mixtures. Plant extracts based on water/propylene glycol in the ratio 1:10 to 10:1 have proven particularly suitable.

According to the invention, the plant extracts can be used in both pure and diluted form. When they are used in diluted form, they normally comprise approximately 2-80 wt. % active substance, and the solvent is the extracting composition or mixture of extracting compositions used for their preparation.

In addition, it can be preferred to employ mixtures of a plurality, particularly two different plant extracts in the compositions according to the invention.

Ii can additionally prove advantageous when the inventive compositions comprise penetration aids and/or swelling agents (M). These include, for example, urea and urea derivatives, guanidine and its derivatives, arginine and its derivatives, water glass, imidazole and its derivatives, histidine and its derivatives, benzyl alcohol, glycerine, glycol and glycol ethers, propylene glycol and propylene glycol ethers, for example propylene glycol monoethyl ether, carbonates, hydrogen carbonates, diols and triols, and particularly 1,2-diols and 1,3-diols such as for example 1,2-propane diol, 1,2-pentane diol, 1,2-hexane diol, 1,2-dodecane diol, 1,3-propane diol, 1,6-hexane diol, 1,5-pentane diol, 1,4-butane diol.

In the context of the invention short chain carboxylic acids (N) can, in addition, advantageously support the complex of active substances (A). In the context of the invention, short chain carboxylic acids and their derivatives are understood to mean carboxylic acids that can be saturated or unsaturated and/or linear or branched or cyclic and/or aromatic and/or heterocyclic and have a molecular weight of less than 750. In the context of the invention, saturated or unsaturated linear or branched carboxylic acids with a chain length of 1 to 16 carbon atoms in the chain can be preferred, those with a chain length of 1 up to 12 carbon atoms in the chain are quite particularly preferred.

In the context of the invention, the short chain carboxylic acids can have one, two, three or more carboxyl groups. In the context of the invention, carboxylic acids with a plurality of carboxyl groups are preferred, particularly di and tricarboxylic acids. The carboxyl groups can be totally or partially present as esters, acid anhydrides, lactones, amides, imide acid, lactams, lactims, dicarboximides, carbohydrazide, hydrazone, hydroxams, hydroxims, amidines, amidoximes, nitriles, phosphonate- or phosphate esters. The inventive carboxylic acids can of course be substituted along the carbon chain or on the cyclic structure. The substituents of the inventive carboxylic acids include, for example C1-C8-alkyl-, C2-C8-alkenyl-, aryl-, aralkyl- and aralkenyl-, hydroxymethyl-, C2-C8-hydroxyalkyl-, C2-C8-hydroxyalkenyl-, aminomethyl-, C2-C8-aminoalkyl-, cyano-, formyl-, oxo-, thioxo-, hydroxy-, mercapto-, amino-, carboxyl- or imino groups. Preferred substituents are C1-C8-alkyl, hydroxymethyl, hydroxy, amino and carboxyl groups. Substituents in the α-position are particularly preferred. Quite particularly preferred substituents are hydroxy-, alkoxy- and amino groups, wherein the amino function can be optionally further substituted by alkyl, aryl, aralkyl and/or alkenyl groups. In addition, equally preferred carboxylic acid derivatives are the phosphonate and phosphate esters.

The silicones represent a particularly preferred group of ingredients.

Preferred inventive agents comprise at least one silicone, preferably a silicone selected from:
(i) volatile or non-volatile, linear or cyclic, crosslinked or non-crosslinked polyalkylsiloxanes, polyarylsiloxanes, polyalkylarylsiloxanes,
(ii) polysiloxanes, which comprise one or more organofunctional groups in their general structure, selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and/or
   m) sulfate or thiosulfate groups;
(iii) linear polysiloxane(A)-polyoxyalkylene(B) block copoylmers of the type (A-B)ₙ with n>3;
(iv) grafted silicone polymers with non silicone-containing organic structures that consist of an organic backbone that is formed from organic monomers that do not comprise silicone, on which in the chain as well as optionally on at least one chain end at least one polysiloxane macromer has been grafted; and/or
(v) grafted silicone polymers with polysiloxane backbone, grafted onto the non silicone-containing organic monomer, which possess a polysiloxane main chain on which in the chain as well as optionally on at least one chain end at least one organic macromer has been grafted that comprises no silicone.

Particularly preferred inventive compositions comprise the silicone(s) preferably in quantities of 0.1 to 10 wt. %, preferably 0.25 to 7 wt. % and especially from 0.5 to 5 wt. %, each based on the total composition.

Preferred silicones will be described below.

Particularly preferred inventive agents comprise at least one silicone of the formula Si-I—

(CH₃)₃Si—[O—Si(CH₃)₂]_{K}—O—Si(CH₃)₃ (Si-I)

wherein x represents a number from 0 to 100, advantageously from 0 to 50, more preferably from 0 to 20 and especially 0 to 10.

These silicones are designated as DIMETHICONE according to INCI nomenclature. In the context of the present invention, preferred compounds employed as the silicone of the formula Si-I include—

(CH₃)₃Si—O—(CH₃)₃

(CH₃)₃Si—O—(CH₃)₂Si—O—Si(CH₃)₃

(CH₃)₃Si—[O—(CH₃)₂Si]₂—O—Si(CH₃)₃

(CH₃)₃Si—[O—(CH₃)₂Si]₃—O—Si(CH₃)₃

(CH₃)₃Si—[O—(CH₃)₂Si]₄—O—Si(CH₃)₃

(CH₃)₃Si—[O—(CH₃)₂Si]₅—O—Si(CH₃)₃

(CH₃)₃Si—[O—(CH₃)₂Si]₆—O—Si(CH₃)₃

(CH₃)₃Si—[O—(CH₃)₂Si]—O—Si(CH₃)₃

(CH₃)₃Si—[O—(CH₃)₂Si]—O—Si(CH₃)₃

(CH₃)₃Si—[O—(CH₃)₂Si]₉—O—Si(CH₃)₃

(CH₃)₃Si—[O—(CH₃)₂Si]₁₀—O—Si(CH₃)₃

(CH₃)₃Si—[O—(CH₃)₂Si]₁₁—O—Si(CH₃)₃

(CH₃)₃Si—[O—(CH₃)₂Si]₁₂—O—Si(CH₃)₃

(CH₃)₃Si—[O—(CH₃)₂Si]₁₃—O—Si(CH₃)₃

(CH₃)₃Si—[O—(CH₃)₂Si]₁₄—O—Si(CH₃)₃

(CH₃)₃Si—[O—(CH₃)₂Si]₁₅—O—Si(CH₃)₃

(CH₃)₃Si—[O—(CH₃)₂Si]₁₆—O—Si(CH₃)₃

(CH₃)₃Si—[O—(CH₃)₂Si]₁₇—O—Si(CH₃)₃

(CH₃)₃Si—[O—(CH₃)₂Si]₁₈—O—Si(CH₃)₃

(CH₃)₃Si—[O—(CH₃)₂Si]₁₉—O—Si(CH₃)₃

(CH₃)₃Si—[O—(CH₃)₂Si]₂₀—O—Si(CH₃)₃

wherein (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are particularly preferred.

Naturally, mixtures of the above cited silicones can also be comprised in the inventive compositions.

Preferred inventively employable silicones have viscosities at 20° C. of 0.2 to 2 mm²s⁻¹, wherein silicones with viscosities of 0.5 to 1 mm²s⁻¹ are particularly preferred.

Particularly preferred agents according to the invention comprise one or more aminofunctional silicones. Such silicones can be described by the Formula—

M(RₐQ_{b}SiO_{(4-a-b)/2)x}(R_{c}SiO_{(4-c)/2})_{y}M

wherein, in the above formula R is a hydrocarbon or a hydrocarbon group with 1 to 6 carbon atoms; Q is a polar group of the general formula —R¹HZ, wherein R¹ is a divalent, linking group that is bonded to hydrogen and the group Z, made up of carbon atoms and hydrogen atoms, carbon-, hydrogen- and oxygen atoms or carbon-, hydrogen- and nitrogen atoms; and Z is an organic amino functionalized group that comprises at least one amino functional group; “a” assumes values in the range of about 0 to about 2, “b” assumes values in the range of about 1 to about 3, “a”+“b” is less than or equal to 3, and “c” is a number in the range of about 1 to about 3, and x is a number in the range of 1 to about 2000, advantageously from about 3 to about 50 and most preferably from about 3 to about 25, and y is a number in the range of about 20 to about 10 000, advantageously from about 125 to about 10 000 and most preferably from about 150 to about 1000, and M is a suitable silicone end-group, as is known from the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl and the like; alkenyl groups, such as vinyl, halogenovinyl, alkylvinyl, allyl, halogenoallyl, alkylallyl; cycloalkyl groups, such as cyclobutyl, cyclopentyl, cyclohexyl and the like; phenyl groups, benzyl groups, halogenated hydrocarbon groups, such as 3-chloropropyl, 4-bromobutyl, 3,3,3-trifluoropropyl, chlorocyclohexyl, bromophenyl, chlorophenyl and the like as well as sulfur-containing groups, such as mercaptoethyl, mercaptopropyl, mercaptohexyl, mercaptophenyl and the like; advantageously R is an alkyl group that comprises 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino functional group comprising at least one functional amino group. A possible formula for Z is NH(CH₂)_{z}NH₂, wherein z is 1 or more. Another possible formula for Z is —NH(CH₂)_{z}(CHCH₂)_{zz}NH, wherein both z and zz independently are 1 or more, wherein this structure includes diamino ring structures, such as piperazinyl. Most preferably, Z is an —NHCH₂CH₂NH₂ group. Another possible formula for Z is —N(CH₂)_{z}(CH₂)_{zz}NX₂ or —NX₂, in which each X of X₂ is independently selected from the group consisting of hydrogen and alkyl groups with 1 to 12 carbon atoms, and zz is 0.

Most preferably, Q is a polar, amine functional group of formula —CH₂CH₂CH₂NHCH₂CH₂NH₂. In the formulae “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)/2} units is in the range from about 1:2 to 1:65, preferably from about 1:5 to about 1:65 and most preferably from about 1:15 to about 1:20. If one or a plurality of silicones of the above formula are added, then the different variable substituents in the above formula for the different silicone components that are present in the silicone mixture can be different.

Preferred inventive agents comprise an amino-functional silicone of Formula (Si-II)—

R′ₐG₃₋ₐ-Si(OSiG₂)ₙ-(OSiG_{b}R′_{2-b})ₘ—O—SiG₃₋ₐ-R′ₐ (Si-II)

wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between 0 and 3, particularly 0;
b is a number between 0 and 1, particularly 1,
m and n are numbers whose sum (m+n) is from 1 to 2000, preferably from 50 to 150, wherein n preferably assumes values of 0 to 1999 and particularly 49 to 149, and m preferably assumes values of 1 to 2000, particularly 1 to 10,
R′ is a monovalent group chosen from
   -Q-N(R″)—CH₂—CH₂—N(R″)₂,
   -Q-N(R″)₂
   -Q-N⁺(R″)₃A⁻
   -Q-N⁺H(R″)₂A⁻
   -Q-N⁺H₂(R″)A⁻ and
   -Q-N(R″)—CH₂—CH₂—N⁺R″H₂A⁻
wherein each Q stands for a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—, and
R″ stands for the same or different groups from the group —H, -phenyl, -benzyl, —CH₂—CH(CH₃)Ph, the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion that is preferably selected from chloride, bromide, iodide or methosulfate.

Particularly preferred inventive agents comprise at least one amino-functional silicone of Formula (Si-IIa)—

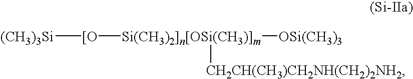

wherein m and n are numbers whose sum (m+n) is from 1 to 2000, preferably from 50 to 150, wherein n preferably assumes values of 0 to 1999 and particularly 49 to 149, and m preferably assumes values of 1 to 2000, particularly 1 to 10.

These silicones are designated as Trimethylsilylamodimethicones according to INCI nomenclature.

Particularly preferred inventive agents are also those that comprise at least one amino-functional silicone of Formula (Si-IIb)—

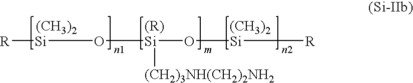

wherein R represents —OH, —O—CH₃ or a —CH₃ group; and m, n1, and n2 are numbers whose sum (m+n1+n2) is from 1 to 2000, preferably from 50 to 150, wherein the sum (n1+n2) preferably assumes values of 0 to 1999 and particularly 49 to 149, and m preferably assumes values of 1 to 2000, particularly 1 to 10.

These silicones are designated as Amodimethicones according to INCI nomenclature.

Independently of which aminofunctional silicones are added, inventive hair conditioners are preferred that comprise an aminofunctional silicone whose amine number is above 0.25 meq/g, preferably above 0.3 meq/g and particularly above 0.4 meq/g. The amine number stands for the milliequivalents of amine per gram of aminofunctional silicone. It can be measured by titration and is also reported with the unit mg KOH/g.

According to the invention, preferred agents comprise, based on their weight, 0.01 to 10 wt. %, preferably 0.1 to 8 wt. %, particularly preferably 0.25 to 7.5 wt. % and particularly 0.5 to 5 wt. % aminofunctional silicone(s).

Also, according to the invention, the addition of cyclic dimethicones, designated by INCI as CYCLOMETHICONES, is preferred. Here, inventive agents are preferred that comprise at least one silicone of Formula (Si-III)—

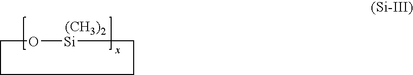

wherein x represents a number from 3 to 200, advantageously from 3 to 10, more preferably from 3 to 7 and especially 3, 4, 5 or 6.

The above described silicones possess a backbone that is constructed from —Si—O—Si— units. Of course, these Si—O—Si units can also be interrupted by carbon chains. Appropriate molecules are obtained by chain extension reactions and are preferably employed in the form of silicone in water emulsions.

Likewise preferred inventive agents comprise at least one silicone of Formula (Si-IV)—

R₃Si—[O—SiR₂]ₓ—(CH₂)ₙ—[O—SiR₂]_{y}—O—SiR₃ (Si-IV)

wherein R represents the same or different groups from the group —H, -phenyl, -benzyl, —CH₂—CH(CH₃)Ph, the C₁₋₂₀ alkyl groups, preferably —CH₃, CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃; x or y represent a number from 0 to 200, preferably from 0 to 10, more preferably from 0 to 7 and especially 0, 1, 2, 3, 4, 5 or 6; and n stands for a number from 0 to 10, preferably from 1 to 8 and particularly for 2, 3, 4, 5, 6.

The silicones are preferably water-soluble. Inventively preferred agents comprise at least one water-soluble silicone.

“Clear” products are often preferred by consumers on esthetic grounds. Accordingly, inventively preferred agents are therefore transparent or translucent.

In the context of the present invention a composition is understood to be transparent or translucent when its NTU value is below 100. The NTU-unit (Nephelometry Turbidity Unit; NTU) is a unit used in water treatment for turbidity measurements in liquids. It is the unit of turbidity of a liquid measured with a calibrated nephelometer.

The inventive agents possess advantageous characteristics and likewise confer advantageous characteristics to hair treated with said agents. Advantages were particularly observed for hair and scalp treatment. Thus, inventive hair treatment agents augment the elasticity of hair treated with them and lead to a strengthening of the internal structure of the hair fibers, which is reflected, for example in higher melting temperatures in differential thermal analyses, in a significantly increased volume and in a higher hold of the style for a longer period.

It also demonstrates an improvement in wet and dry combability as well as preventing a premature formation of split ends for the treated hair.

A further subject matter of the invention is the use of mixtures of:
a) at least one chitosan and/or chitosan derivative and
b) at least one elastomeric silane or siloxane containing quaternary ammonium groups in the molecule
   in order to improve at least one of the following properties—
tensile strength of keratinic fibers, especially human hair;
stabilization of the moisture balance of keratinic fibers, especially human hair;
combability of keratinic fibers, especially human hair;
increased volume of keratinic fibers, especially human hair;
slowing down the aging process of keratinic fibers, especially human hair; and
reducing the fall-off in elasticity of keratinic fibers, especially human hair, from damage by atmospheric action.

As a result of the addition of the special combination of active substances, the inventive agent also possesses these characteristics. Accordingly, a further subject matter of the invention is the use of the inventive hair treatment agent for improving at least one of the characteristics

With reference to further preferred embodiments of the use according to the invention, the statement made concerning the agents according to the invention applies mutatis mutandis.

### EXAMPLE

Unless otherwise stated, the quantities are understood to be in weight percent. The following formulation was produced—

| Raw material | |
|---|---|
| Benzophene-4 | 0.05 |
| Citric acid | 0.01 |
| Kytamer PC¹ | 1.00 |
| Ethanol | 15.00 |
| Dow Corning 5-7070 emulsion² | 5.00 |
| D-Panthenol | 0.25 |
| PEG-40 hydrogenated castor oil | 0.50 |
| Perfume | 0.10 |
| Water | ad 100 |

¹Chitosonium pyrrolidone carboxylate, reaktion product of chitosan with pyrrolidone carboxylic acid, powder comprising 77 to 87 wt. % active substance (INCI name: Chitosan PCA) (Amerchol Corp.)
²Silicone emulsion, ca. 27 wt. % silicone elastomer active substance (INCI name: Silicone Quaternium-16/Glycidoxy Dimethicone Crosspolymer, Trideceth-12) (Dow Corning)

On using the composition on human hair, an excellent, long lasting shape stabilization was achieved. The hair was full and voluminous.

We claim:

1. Hair treatment agent comprising:
0.05 to 5 wt. % of at least one chitosan and/or chitosan derivative, and
0.05 to 5 wt. % of at least one elastomeric silane or siloxane having quaternary ammonium groups in the molecule,
each based on total weight of the hair treatment agent.

2. Hair treatment agent according to claim 1, wherein the chitosan derivative(s) is/are neutralization products of chitosan with lactic acid, pyrrolidone carboxylic acid, nicotinic acid, hydroxy-iso-butyric acid, hydroxy-iso-valeric acid or mixtures thereof.

3. Hair treatment agent according to claim 1 further comprising 0.1 to 4.5 wt. %, based on total weight of the agent, a neutralization product of chitosan with pyrrolidone carboxylic acid.

4. Hair treatment agent according to claim 1, wherein the elastomeric silane(s) or siloxane(s) having quaternary ammonium groups in the molecule are in the form of an oil-in-water (O/W) or water-in-oil (W/O) emulsion or microemulsion, and wherein the silane or siloxane forms the oil phase.

5. Hair treatment agent according to claim 1, wherein the elastomeric silane(s) or siloxane(s) having quaternary ammonium groups in the molecule are obtained from the reaction of an organic quaternary ammonium compound containing epoxide groups or halohydrin groups, with a silane or siloxane containing amino groups in the presence of a branching agent, and a surfactant, dispersed in an aqueous polar phase.

6. Hair treatment agent according to claim 5 wherein the organic quaternary ammonium compound containing epoxide groups is chosen from glycidyl-trimethylammonium chloride and/or glycidyl-trimethylammonium bromide.

7. Hair treatment agent according to claim 5, wherein the organic quaternary ammonium compound containing halohydrin groups is chosen from (3-chloro-2-hydroxypropyl)trimethylammonium chloride, (3-chloro-2-hydroxypropyl)dimethyldodecylammonium chloride, (3-chloro-2-hydroxypropyl)dimethyloctadecylammonium chloride, (3-chloro-2-hydroxypropyl)trimethylammonium bromide, (3-chloro-2-hydroxypropyl)dimethyldodecylammonium bromide, and (3-chloro-2-hydroxypropyl)-dimethyloctadecylammonium bromide.

8. Hair treatment agent according to claim 5, wherein the branching agent is chosen from organic epoxides having at least two epoxide groups, epoxy-functional silicones having at least two epoxide groups, chlorohydrins, substituted di(meth)acrylates, unsubstituted di(meth)acrylates, oligo(meth)acrylates, substituted mono(meth)acrylates, hydroxyalkyl acrylates and isocyanates.

9. Hair treatment agent according to claim 5, wherein the branching agent is chosen from butane diol diglycidyl ether and/or propylene glycol diglycidyl ether.

10. Hair treatment agent according to claim 5, wherein the surfactant is at least a non-ionic surfactant.

11. Hair treatment agent according to claim 10, wherein the non-ionic surfactant is at least an alkoxylated alcohol surfactant.

12. Hair treatment agent according to claim 11, wherein the alkoxylated alcohol surfactant is an ethoxylated alcohol of the formula
CₙH₂ₙ₊₁(OCH₂CH₂)ₓOH
wherein n is a number from 8 to 20, and x is a number from 8 to 18.

13. Hair treatment agent according to claim 4, wherein the emulsion or microemulsion comprises a silicone content of 15 to 40 wt. %, based on total weight of the emulsion or microemulsion.

14. Hair treatment agent according to claim 1, wherein the elastomeric silane(s) or siloxane(s) having quaternary ammonium groups in the molecule exhibit the group
—CH₂CH(OH)CH₂N⁺(CH₃)₃Cl⁻
as the quaternary ammonium group.

15. Hair treatment agent according to claim 1, further comprising 0.5 to 70 wt. %, based on total weight of the agent, of an anionic and/or non-ionic and/or cationic and/or amphoteric surfactant(s).

16. Hair treatment agent according to claim 1, further comprising 0.5 to 7.5 wt. %, based on total weight of the agent, of at least one cationic polymer chosen from poly(methacryloyloxyethyltrimethylammonium chloride) (INCI: Polyquaternium 37) and/or; quaternized cellulose derivatives (INCI: Polyquaternium 10) and/or cationic alkyl polyglycosides and/or cationized honey and/or cationic guar derivatives and/or polymeric dimethyldiallylammonium salts and their copolymers with esters and amides of acrylic acid and methacrylic acid and/or copolymers of vinyl pyrrolidone with quaternized derivatives of dialkylaminoalkyl acrylate and dialkylaminoalkyl methacrylate and/or vinyl pyrrolidone-vinylimidazolium methochloride copolymers and/or quaternized polyvinyl alcohol and/or Polyquaternium 2 and/or Polyquaternium 7 and/or Polyquaternium 17 and/or Polyquaternium 18 and/or Polyquaternium 24 and/or Polyquaternium 27.
